(19) United States
(12) Patent Application Publication
(10) Pub. No.: US 2008/0269463 A1
(43) Pub. Date: Oct. 30, 2008

(54) Tropoelastin Isoforms and Used Thereof

(75) Inventors: Cheryl L. Maslen, Lake Oswego, OR (US); Darcie Babcock, Portland, OR (US)

Correspondence Address: KLARQUIST SPARKMAN, LLP, 121 SW SALMON STREET, SUITE 1600, PORTLAND, OR, 97204, US

(73) Assignee: OREGON HEALTH & SCIENCE UNIVERSITY, Portland, OR (US)

(21) Appl. No.: 11/817,499

(22) PCT Filed: Mar. 3, 2006
(86) PCT No.: PCT/US06/07831

§ 371 Date: Aug. 30, 2007

Related U.S. Application Data
(60) Provisional application No. 60/658,890, filed on Mar. 4, 2005.
(60) Provisional application No. 60/668,361, filed on Apr. 4, 2005.

(51) Int. Cl. C07K 14/00; C07H 21/04

(52) U.S. Cl. 530/353; 536/23.5

(57) ABSTRACT

This disclosure provides new isoforms of tropoelastin. The disclosure further provides methods for making and using these isoforms, alone or in combination with each other or other isomers, such as in the production of biomaterials.

### CROSS-REFERENCE TO RELATED APPLICATIONS

This application claims priority to and benefit of U.S. Provisional Applications 60/658,890, filed Mar. 4, 2005 and 60/668,361 filed Apr. 4, 2005, which are incorporated herein by reference in their entirety.

### STATEMENT OF GOVERNMENT SUPPORT

This invention was made in part with United States government support pursuant to Department of Defense grant no. DAMD17-96-1-6006. The United States government may have certain rights in the invention.

### FIELD

This disclosure relates to the field of recombinant proteins. More specifically, this disclosure concerns nucleic acids encoding novel tropoelastin isoforms, tropoelastin monomers encoded thereby, elastin polymers and methods for their production and use.

### BACKGROUND

Expression of the elastin (ELN) gene produces tropoelastin, a soluble monomeric protein that assembles into elastic fibers (Rosenbloom et al., FASEB Journal 7:1208-1218, 1993). Elastic fibers are extracellular structural components of many elastic tissues and have two structural components, elastin and microfibrils (Gibson et al., J Biol Chem 264:4590-4598, 1989). Microfibrils are complex fibrillar structures containing several proteins including the fibrillins (Kielty et al., Int J Biochem Cell Bio 27:747-760, 1995; Sherratt et al., Micron 32:185-200, 2001). During development, microfibrils appear to be a scaffold onto which elastin is deposited. Microfibrils bind growth factors (Taipale et al., J Histo. Cyto. 44:875-889, 1996), but also contribute to the mechanical properties of the elastic fiber. However, it is elastin that endows the tissue with elasticity and resilience. These properties are important for the functioning of tissues such as arterial vessels, lungs, and skin.

It has been known for many years that tropoelastin is synthesized as a number of isoforms, initially from protein studies (Rich and Foster, BBRC 146:1291-1295, 1987) and later RNA analysis of several tissues and cells from several species (Indik et al., PNAS USA 84:5680-5684, 1987; Raju and Anwar, J Biol Chem 262:5755-5762, 1987; Baule et al., BBRC 154:1054-1060, 1988; Parks et al., Matrix 12:156-162, 1992). Alternate splicing is species dependent (Wrenn et al., J Biol Chem 262:2244-2249, 1987), developmental age dependent (Parks et al., J Biol Chem 263:4416-4423, 1988) and tissue specific (Heim et al., Matrix 11:359-366, 1991). On completion of the human tropoelastin gene sequence (Fazio et al., J Invest Dermatology 91:458-464, 1988), it became apparent that several of the at least 35 exons are expressed to varying degrees but the majority of exons are retained in all tropoelastin mRNAs.

The assumption has persisted that the majority of ELN transcripts represent molecules that contain all of the coding exons (with the exceptions of exons 22 and 26A), and that alternative splicing is a low level event. This was likely based on the observation of a single 3.5 kb band on northern blot analyses of some tissues, although it was recognized early on that the size resolution on standard northern blot analysis could easily miss microheterogeneity of mRNA species (Fazio et al., J. Invest. Dermatol. 91:458-464, 1988). Indeed, there is no evidence for the expression of native full-length tropoelastin utilizing every exon except 26A, a fact that has not been taken into account in studies of the assembly and properties of elastin.

Alternatively spliced tropoelastin mRNAs are translated into protein and incorporated into chick aorta, demonstrating that alternative splicing really affects the composition of the extracellular matrix and is a functioning control mechanism at the protein level (Pollock et al., J Biol Chem 265:3697-3702, 1990). Exon 26A is unusual as it has only been found in isoforms expressed by keratinocytes (Hirano et al., Arch. Dermatol. Res. 293:430-433, 2001) and in pulmonary hypertension by neointima cells (Bisaccia et al., Biochemistry 37:11128-11135, 1998) and in human fetal aorta (Indik et al., PNAS USA 84:5680-5684, 1987). Exons, besides 26A, that are frequently spliced out of human tropoelastin isoforms include 22, 23, and 32. Certain exons with known important biological functions have never been found to be spliced out suggesting that alternative splicing is not a random occurrence. Specific sequence elements in the rat (Pierce et al., Genomics 12:651-658, 1992) and bovine (Yeh et al., Collagen and Related Research 7:235-247, 1987) tropoelastin genes that could signal splicing were identified for many of the alternatively spliced exons thereby revealing possible control mechanisms. The carboxy-terminal exon 36 is always present in native tropoelastin molecules, and may be critical in elastogenesis (Brown-Augsburger et al., J Biol Chem 269:28443-28449, 1994). It is conserved across species and has a unique amino acid sequence that includes the only two cysteine residues in secreted tropoelastin together with a characteristic block of four basic amino acids that form a charged pocket (Brown et al, BBRC 186:549-555, 1992). This exon also contains integrin-binding and other cell-binding sites. Exon 30 mediates elastin deposition (Kozel et al., J Biol Chem 278:18491-18498, 2003). Similarly, exon 24 contains a conserved hexapeptide repeat VGVAPG that has chemotactic activity, and is a sequence that binds to the cell surface elastin-binding protein (Hinek et al, Science 239:1539-1541, 1988; Mecham et al., Biochemistry 28:3716-3722, 1989).

However, all investigations of elastin and tropoelastin protein to date have been made without regard to isoform composition. How different isoforms contribute to elastin function has remained unknown due to significant difficulties associated with biochemical analysis of elastin. Tropoelastin molecules are rapidly cross-linked into an insoluble matrix, making them virtually inaccessible for isolation and characterization. Since mature elastin is a highly insoluble protein made up of crosslinked tropoelastin monomers, it has not been possible to directly determine the isoform content of a mature elastin fiber. Consequently, it has been impossible to determine isoform composition in vivo. It has not been possible to study tropoelastin protein isoforms in vitro because they have not been available in sufficient quantities for investigation. The present disclosure addresses these needs, and provides three novel tropoelastin isoforms with favorable characteristics in the production of elastin biomaterials.

### SUMMARY

This disclosure provides new tropoelastin isoforms, as well as methods for producing and for using these novel tropoelastin molecules. The unique physical and biological properties of these isoforms can be used, for instance, to alter the properties of elastin-based biomaterials to confer desired physical and biological properties on the final product.

The unique tropoelastin isoforms described herein can be used individually or in combination with other tropoelastin isoforms to create new elastin molecules with unique properties that can be advantageously employed in various applications, for example, to improve tensile strength, elasticity, or to alter biological properties such as cell binding or chemotaxicity. Since different biomaterial applications require different physical or biological properties, use of these isoforms individually or in combinations can tailor the biomaterial to a specific need. The foregoing and other objects, features, and advantages will become more apparent from the following detailed description, which proceeds with reference to the accompanying figures.

### BRIEF DESCRIPTION OF THE DRAWINGS

FIG. 1 is a diagrammatic representation of human tropoelastin protein domains. The exon numbering system is based upon the bovine elastin gene sequence. The human gene has no exons 34 and 35, and exon 26A is unique to human tropoelastin.

FIG. 2 is a representation of the amino acid sequence of a human tropoelastin reference isoform including all of the translated exons (SEQ ID NO: 14). The amino acid compositions of tropoelastin isoforms I, J and L (numbered as is the reference isoform) are indicated in the lower panel.

FIG. 3 is a line graph illustrating coacervation curves for two different human tropoelastin isoforms. Isoform A is designated by a diamond (♦) and a square (▪), isoform E is designated by a triangle (▴) and an X.

### SUMMARY OF THE SEQUENCE LISTING

SEQ ID NO:1 is the nucleotide sequence of tropoelastin isoform i cDNA (long form).

SEQ ID NO:2 is the amino acid sequence of tropoelastin isoform I (long form).

SEQ ID NO:3 is the nucleotide sequence of tropoelastin isoform j cDNA (long form).

SEQ ID NO:4 is the amino acid sequence of tropoelastin isoform J (long form).

SEQ ID NO:5 is the nucleotide sequence of tropoelastin isoform 1 cDNA (long form).

SEQ ID NO:6 is the amino acid sequence of tropoelastin isoform L (long form).

SEQ ID NO:7 is the nucleotide sequence of tropoelastin isoform i cDNA (short form).

SEQ ID NO:8 is the amino acid sequence of tropoelastin isoform I (short form).

SEQ ID NO:9 is the nucleotide sequence of tropoelastin isoform j cDNA (short form).

SEQ ID NO:10 is the amino acid sequence of tropoelastin isoform J (short form).

SEQ ID NO:11 is the nucleotide sequence of tropoelastin isoform 1 cDNA (short form).

SEQ ID NO:12 is the amino acid sequence of tropoelastin isoform L (short form).

SEQ ID NO:13 is the nucleotide sequence of a reference human tropoelastin isoform.

SEQ ID NO:14 is the amino acid sequence of a human tropoelastin reference isoform.

SEQ ID NO:15 is the amino acid sequence encoded by exon 7A.

SEQ ID NO:16 is the amino acid sequence encoded by exon 23A.

### DETAILED DESCRIPTION

The human elastin gene (ELN), which encodes the protein tropoelastin, is subject to alternative splicing. However, the number of splice variants has never been determined, and not all coding exons have been characterized. Characterization of the ELN transcriptome led to the discovery of three previously undescribed alternative splice junctions, which when utilized add three new exons to the molecular repertoire of ELN. Inclusion of these exons occurs as the result of activation of otherwise cryptic donor or acceptor splice junctions that can be used in place of the corresponding constitutive junction. Alternate use of neighboring cryptic splice junctions is often seen as a result of mutation, but in ELN it appears to be a mechanism of normal alternative splicing rather than a failure to recognize the appropriate splice site. Identification of these exons demonstrates that ELN has 37 coding exons as opposed to the 34 previously validated exons. Out of these 37 exons, 17 are alternatively spliced. Nearly all of the alternatively spliced exons are skipped in more than one isoform transcript, and usually in more than one tissue. The extensive variability of transcripts results in the potential for the expression of a large number of tropoelastin isoforms, some of which are tissue specific. Hence, alternative splicing of ELN leads to the phenotypic variability of elastic structures.

Splicing affects a number of distinct properties of tropoelastin, and consequently of elastin polymers made therefrom. For example, although the mechanism of tropoelastin assembly into elastic fibers is poorly understood, modification of the exon composition is likely to alter the assembly properties of elastin (e.g., by altering coacervation, cross-linking, or both). In addition, introducing a small proportion of different tropoelastin isoforms into a growing polymer is likely to affect the mechanical and biological properties of the elastin produced. Some exons contain binding sites for specific proteins and cells, such that presence or absence of a particular exon modifies or eliminates these interactions, which can influence the deposition of tropoelastin onto a growing elastin fiber and/or during the tissue remodeling process.

Through a combination of cloning and characterizing transcriptional products from a fetal heart library, and interrogation of public gene sequence databases, 19 tropoelastin splice variants were identified and validated. Of the 19 variants identified, three were previously unknown.

Thus, one aspect of the disclosure relates to an isolated or recombinant nucleic acid, which includes a polynucleotide sequence that encodes a novel human tropoelastin isoform. The novel tropoelastin isoform nucleic acids include exon 20A, and exclude one or more of exons 3, 7A, 13, 19, 22, 23, 23A, 26A and/or 32 of the human elastin (ELN) genomic sequence (the ELN gene). For example, the nucleic acids can exclude a combination of exons, such as (a) exons 7A, 13, 22, 23, 23A and 26A; (b) exons 7A, 19, 22, 23A, 26A, and 32; or (c) exons 3, 7A, 22, 23, 23A, 26A, and 32. With respect to exon composition, such nucleic acids can be selected from the following group: (a) a polynucleotide sequence including exons 2, 3, 4, 5, 6, 7, 8, 9, 10, 11, 12, 14, 15, 16, 17, 18, 19, 20, 20A, 21, 24, 25, 26, 27, 28, 29, 30, 31, 32, 33, and 36; (b) a polynucleotide sequence including exons 2, 3, 4, 5, 6, 7, 8, 9, 10, 11, 12, 13, 14, 15, 16, 17, 18, 20, 20A, 21, 23, 24, 25, 26, 27, 28, 29, 30, 31, 33, and 36; (c) a polynucleotide sequence including exons 2, 4, 5, 6, 7, 8, 9, 10, 11, 12, 13, 14, 15, 16, 17, 18, 19, 20, 20A, 21, 24, 25, 26, 27, 28, 29, 30, 31, 33, and 36 of the human ELN genomic sequence; and (d) the polynucleotide sequence of (a), (b), or (c) with the 5′ addition of exon 1 of the human ELN genomic sequence.

In exemplary embodiments, the isolated or recombinant nucleic acid includes a polynucleotide sequence that encodes a polypeptide with the amino acid sequence of SEQ ID NO:2; SEQ ID NO:4, SEQ ID NO:6, SEQ ID NO:8, SEQ ID NO:10, or SEQ ID NO:12 or a variant thereof having no more than 10 (e.g., 1, 2, 3, 4, 5, etc.) amino acid deletions, additions or conservative amino acid substitutions. In certain embodiments, the isolated or recombinant nucleic acid encodes a polypeptide that includes no more than 1 amino acid deletion, addition or conservative amino acid substitution.

For example, the isolated or recombinant nucleic acid encoding a tropoelastin isoform can be a polynucleotide sequence selected from the group consisting of SEQ ID NO:1, SEQ ID NO:3, SEQ ID NO:5, SEQ ID NO:7, SEQ ID NO:9 or SEQ ID NO:11, and a variant thereof, which variant hybridizes under high stringency conditions over substantially the entire length to one or more of SEQ ID NOs:1, 3, 5, 7, 9 and/or 11. In particular embodiments, the isolated or recombinant nucleic acid is SEQ ID NO:1; SEQ ID NO:3, SEQ ID NO:5, SEQ ID NO:7, SEQ ID NO:9 or SEQ ID NO:11.

Another aspect of the disclosure relates to novel isolated tropoelastin isoform monomers. The isolated tropoelastin monomers are the products of alternative splicing of the ELN genomic sequence and exclude the amino acids encoded by (a) exons 7A, 13, 22, 23, 23A and 26A; (b) exons 7A, 19, 22, 23A, 26A, and 32; or (c) exons 3, 7A, 22, 23, 23A, 26A, and 32. For example, the novel tropoelastin monomer can consist of the amino acids encoded by: (a) a polynucleotide sequence including exons 2, 3, 4, 5, 6, 7, 8, 9, 10, 11, 12, 14, 15, 16, 17, 18, 19, 20, 20A, 21, 24, 25, 26, 27, 28, 29, 30, 31, 32, 33, and 36; (b) a polynucleotide sequence including exons 2, 3, 4, 5, 6, 7, 8, 9, 10, 11, 12, 13, 14, 15, 16, 17, 18, 20, 20A, 21, 23, 24, 25, 26, 27, 28, 29, 30, 31, 33, and 36; or (c) a polynucleotide sequence including exons 2, 4, 5, 6, 7, 8, 9, 10, 11, 12, 13, 14, 15, 16, 17, 18, 19, 20, 20A, 21, 24, 25, 26, 27, 28, 29, 30, 31, 33, and 36 of the human ELN genomic sequence.

Exemplary tropoelastin monomers are represented by SEQ ID NO:2; SEQ ID NO:4, SEQ ID NO:6, SEQ ID NO:8, SEQ ID NO:10 and SEQ ID NO:12. Variant thereof having no more than 10 amino acid deletions, additions or conservative amino acid substitutions are also encompassed by this disclosure. For example, in certain embodiments, the tropoelastin monomer includes no more than 1 amino acid deletion, addition or conservative amino acid substitution as compared to SEQ ID NO:2; SEQ ID NO:4, SEQ ID NO:6, SEQ ID NO:8, SEQ ID NO:10 or SEQ ID NO:12.

In certain embodiments, the tropoelastin monomer can be encoded by a nucleic acid comprising the polynucleotide sequence of SEQ ID NO:1, SEQ ID NO:3, SEQ ID NO:5, SEQ ID NO:7, SEQ ID NO:9, SEQ ID NO:11 or a variant thereof, which variant hybridizes under high stringency conditions over substantially the entire length to one or more of SEQ ID NO:1, SEQ ID NO:3, SEQ ID NO:5, SEQ ID NO:7, SEQ ID NO:9, or SEQ ID NO:11.

Another aspect of the disclosure relates to synthetic elastin polymers including one or more of the novel tropoelastin monomers. In some instances, the synthetic elastin polymers include more than one tropoelastin isoform, at least one of which is I, J or L. For example, the synthetic elastin polymer can include one (or two or three) of the novel tropoelastin isoforms (I, J, and/or L) disclosed herein. Optionally, the synthetic elastin polymer can also include one or more previously described tropoelastin monomer, such as any one of the other isoforms shown in Table 2 (e.g., isoform A) that exhibits desirable structural or functional properties.

For example, by virtue of including the one or more novel tropoelastin isoform monomers, the synthetic elastin polymer can exhibit altered coacervation, altered cross-linking, or both altered coacervation and altered cross-linking as compared to an elastin polymer lacking the at least one tropoelastin monomer, such as a synthetic elastin polymer made up of isoform A monomers. Similarly, the synthetic elastin polymer can possess at least one improved biological or functional property as compared to an elastin polymer lacking the at least one tropoelastin monomer, such as a synthetic elastin polymer made up of tropoelastin isoform A. For example, the synthetic elastin polymer can exhibit one or more of the following properties: increased or decreased tensile strength, increased or decreased elasticity, increased or decreased chemotaxicity, and increased or decreased cell-binding, as compared to an elastin polymer lacking the at least one tropoelastin monomer.

Thus, the present disclosure concerns novel nucleic acids, novel proteins and synthetic biomaterials produced therefrom, as well as methods for producing and using the same. Additional technical details are provided under the specific topic headings below.

### Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described herein. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of specific terms are provided:

“Tropoelastin” is a monomeric protein encoded by the elastin (ELN) genomic sequence (or gene). Tropoelastin monomers are approximately 70 kDa in size and include glycine-, valine- and leucine-rich hydrophobic domains interspersed with lysine and alanine containing cross-linking domains. Multiple tropoelastin isoforms are produced by translation of alternatively spliced transcripts of the ELN genomic sequence. Cross-linking of tropoelastin monomers produces the polymer “elastin,” a component of the extracellular matrix of elastic tissues, including lung, blood vessels, heart and skin. The human ELN genomic sequence maps to the long arm of chromosome 7 (7q11.23), and mutations in this locus have been identified in patients with Williams-Beuren syndrome (WBS) and supravalvular aortic stenosis (SVAS). The genomic sequence of the ELN gene is represented by nucleotides 25,442-65,954 of GENBANK® Accession No. AAC005056 (version 2, published Jan. 31, 2004).

“Elastin” or an “elastin polymer” is a polymer made up of cross-linked tropoelastin monomers. The elastin polymer can include one or more than one tropoelastin isoforms. The modifier “synthetic” with respect to an elastin polymer indicates that the polymer is produced in vitro, from isolated and/or recombinant tropoelastin monomers, or that the elastin polymer is produced in vivo following expression of a heterologous (e.g., recombinant) nucleic acid. In vitro production of a synthetic elastin polymer includes, for example, coacervation and cross-linking in vitro, following the production and purification of recombinant tropoelastin monomers from bacterial, yeast, insect or mammalian cells. Production of a synthetic elastin polymer also includes the production of an elastin polymer in vitro from isolated desegregated tropoelastin monomers from a tissue of a multicellular organism, including a transgenic organism that expresses a recombinant (such as, a human) ELN genomic sequence or cDNA.

“Coacervation” is the aggregation of a solute to form solutions that differ in composition or concentration of solutes. Tropoelastin monomers in an aqueous solution coacervate upon heating to between approximately 35 and 42° C., resulting in a highly concentrated (>about 70%) tropoelastin solution and an aqueous equilibrium solution. The elastin coacervate is substantially denser than the equilibrium solution and can be separated by centrifugation or other preparatory methods (such as, filtration).

“Cross-linking” refers to the covalent association between moieties, such as amino acids in a polypeptide. For example, tropoelastin is covalently cross-linked via oxidative deamination and spontaneous condensation of lysine. Cross-linking can be catalyzed enzymatically, e.g. by lysyl oxidases, or chemically, e.g., by cross-linking agents such as bis(sulfosuccinimidyl)suberate (BSS).

The terms “polynucleotide” and “nucleic acid sequence” refer to a polymeric form of nucleotides at least 10 bases in length. Nucleotides can be ribonucleotides, deoxyribonucleotides, or modified forms of either nucleotide. The term includes single and double forms of DNA. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. In one embodiment, a polynucleotide encodes a polypeptide.

An “exon” is a polynucleotide sequence in a nucleic acid that encodes information for protein synthesis.

The term “polypeptide” refers to a polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “polypeptide fragment” refers to a portion of a polypeptide. The term “functional fragments of a polypeptide” refers to all fragments of a polypeptide that retain an activity of the polypeptide.

A “variant” when referring to a nucleic acid or a protein (e.g. an ELN or tropoelastin nucleic acid or protein variant) is a nucleic acid or a protein that differs from a reference nucleic acid or protein. Usually, the difference(s) between the variant and the reference nucleic acid or protein constitute a proportionally small number of differences as compared to the reference. Thus, a variant typically differs by no more than about 1%, or 2%, or 5%, or 10%, or 15%, or 20% of the nucleotide or amino acid residues. For example, a variant tropoelastin cDNA can include 1, or 2, or 5 or 10, or 15, or 50 or up to about 100 nucleotide differences. A variant tropoelastin monomer can include 1, or 2, or 5, or 10, or up to about 30 amino acid differences.

An “isolated” biological component (such as a nucleic acid molecule, protein or organelle) has been substantially separated or purified away from other biological components in the cell of the organism in which the component naturally occurs, such as, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

The term “purified” does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid or protein can be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid or protein content of the preparation.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the microorganism chromosome. The recombinant protein can be heterologous to the microorganism or homologous to the microorganism. As used herein, the term “heterologous protein” refers to a protein or polypeptide that does not naturally occur in a gram-positive host cell.

### Tropoelastin Structure and Function

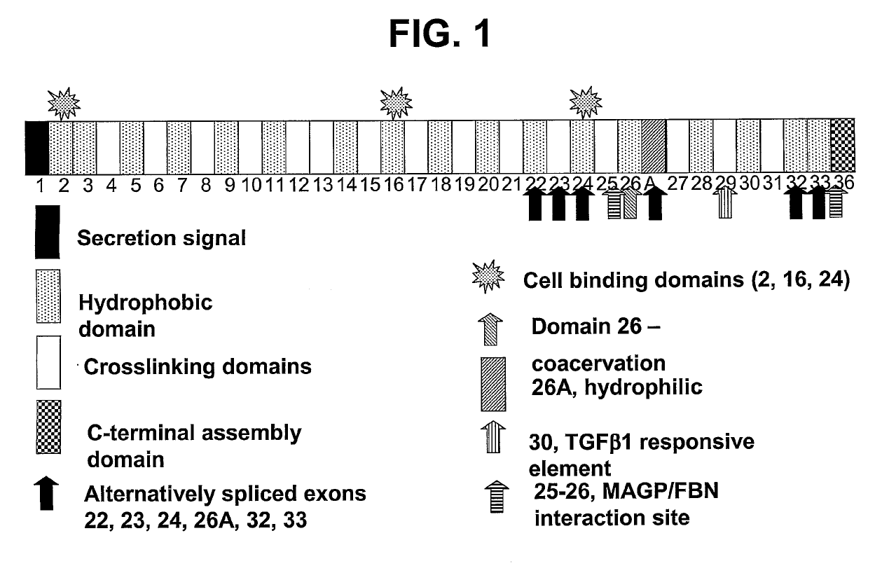

Tropoelastin is an approximately 70 kDa protein that has no posttranslational modifications except a very low and variable level of proline hydroxylation which has no known biological significance. The human tropoelastin gene is composed of at least 35 exons that code either hydrophobic domains rich in glycine, valine and leucine or cross-linking hydrophilic domains containing lysine and alanine (Bashir et al., J Biol Chem 264:8887-8891, 1989). Each exon codes a protein domain that is referred to by its corresponding exon number. These domains are illustrated schematically in FIG. 1. Exon 26A is a unique non-cross-linking hydrophilic domain. The amino terminal domain (Exon 1) contains the signal sequence and carboxy terminal domain (Exon 36) contains a unique cysteine-containing domain that has special functions.

### Coacervation

The primary structure of tropoelastin enables the monomers to self-assemble in vitro at physiological pH, temperature and ionic strength. This phenomenon is called coacervation (Vrhovski et al., Euro J Biochem 250:92-98, 1997). When a solution of tropoelastin is warmed to 37° C., the molecules spontaneously associate to form filamentous structures, which separate out into a dense phase that can be sedimented by centrifugation and isolated from the bulk of the liquid. The role coacervation plays in the in vivo assembly of tropoelastin remains unclear.

Several studies using synthetic peptides based upon tropoelastin exon sequences have shown that coacervation is very sensitive to hydrophobic domain sequence, size and position (see, e.g., Toonkool et al., J Biol Chem 276:44575-44580, 2001). For example, moving domain 26 from its normal position to the carboxy-terminus of the molecule increased the coacervation temperature by 9° C., suggesting that the context or position of domains is also important, not just their presence. Coacervation studies of recombinant peptides representing the carboxy-terminal one-quarter of the tropoelastin molecule (exons 26-34) showed that domain 26 is important in the initial association of tropoelastin molecules (Jensen et al., J Biol Chem 275:28449-28454, 2000). Furthermore, a natural desmosine cross-link has been located between exon 25 and 19 in antiparallel molecules (Brown-Augsburger et al., J Biol Chem 270:17778-17783, 1995). This would place domain 26 adjacent to hydrophobic domain 18. Deleting domain 18 from full-length tropoelastin increased its coacervation temperature by 8° C., whereas deleting domain 26 increased it by 11° C., indicating that these domains are involved in the coacervation process.

Cross-Linking

In order to form cross-links in vivo, tropoelastin molecules are aligned so that lysine residues are juxtaposed. The hydrophobic domains endow elastin with properties of an elastic polymer and are involved in the initial alignment of tropoelastin molecules during the formation of elastin fibers (Bellingham et al., Biochimica et Biophysica Acta 1550:6-19, 2001). The cross-linking domains contain lysine residues that are used to form stable covalent cross-links between the tropoelastin molecules within the elastin fibers (Sakura et al, J. Am. Chem. Soc. 92:3778-3782, 1984). Crosslink-formation is initiated by lysyl oxidase that oxidatively deaminates one of the lysine residues to form an aldehyde group (Kagan and Li, J Cell Biochem 88:660-672, 2003). This aldehyde group spontaneously reacts with a neighboring amino group to form a bi-functional Schiff's base cross-link. Further additions take place, which result in the formation of elastin-specific desmosine and isodesmosine stable cross-links. The exact mechanism of molecular alignment is poorly understood but is thought to initially involve interaction of exon 30 of tropoelastin with fibrillin (Kozel et al., J Biol Chem 278:18491-18498, 2003). Subsequently a self-assembly process, similar to the process of coacervation that can be carried out in vitro (Bressan et al., J Ultrastructural Res 82:335-340, 1983), adds more tropoelastin to the fibrillin-associated molecules. Coacervation causes the formation of filaments, which when treated with lysyl oxidase desmosine and isodesmosine form cross-links (Bedel-Hogan et al, J Biol Chem 268:10345-10350, 1993).

The spacing between cross-linking sites varies in different isoforms, which results in varied alignments of lysine residues in an isoform mixture, causing the density and type of cross-linking to vary with the isoform composition. Therefore, the fabrication of tissue structures is likely influenced by the spectrum of tropoelastin isoforms a particular cell synthesizes. For example, elastin fibers in blood vessels (Crissman et al., Antatomical Record 198:581-593, 1980), lung (Karrer, J Ultrastructural Res 2:96-121, 1957), skin (Weinstein and Boucek, J Investigative Dermatology 35:227-229, 1960) and cartilage (Hesse, Cell and Tissue Res 248:589-593, 1987) have different morphologies, reflecting the physiological function they perform. The synthesis of tissue specific and developmentally regulated isoform mixtures enable specific cell-tropoelastin, protein-tropoelastin and tropoelastin-tropoelastin interactions that lead to the required structures.

### Alternative Splicing of Tropoelastin

ELN transcript variability is more extensive than previously recognized. Alternative splicing of tropoelastin has been shown to be developmentally regulated with the pattern of transcripts changing over time (Parks et al., J. Biol. Chem. 263:4416-4423, 1988). Analysis of splicing in different animals demonstrated that exon skipping patterns are a species-specific phenomenon (Fazio et al., J. Invest. Dermatol. 91:458-464, 1988). This supports the premise that the elastic tissues of other species have specific requirements for elastin composition. Consequently it is not possible to directly correlate the data from previous studies of tropoelastin alternative splicing in other species to the alternative splicing seen in human tissues. However, the commonality of the phenomenon of tropoelastin alternative splicing further suggests an important role for tropoelastin isoforms in tissue characteristics.

The data presented here demonstrate that extensive alternative splicing of ELN is the more common event, with the so called “full-length” transcript (omitting solely exons 22 and 26A) being in the minority, at least in most tissues. Thus, most elastin-expressing cells and tissues are actually synthesizing a mixture of tropoelastin isoforms.

The fact that some exons are apparently not subject to alternative splicing (that is, certain exons are found in all native tropoelastin isoforms) indicates that exon skipping is not a random event, and that some exons contribute sequences that are involved in determining elastin structure and/or function. For example, an essential elastin assembly domain was localized to exons 29-36, with exon 30 identified as encoding a major functional element (Kozel et al., J Biol Chem 278:18491-18498, 2003). Consistent with this is the observation that exon 30 does not appear to be differentially spliced. However, exons 32 and 33 are alternatively spliced and are sometimes both missing from the same transcript, suggesting that they do not contain information essential to elastogenesis.

In some cases, exon composition is tissue specific. For example, exon 26A is usually skipped, but is always included in the primary ELN transcript expressed by terminally differentiated keratinocytes (Hirano et al., Arch. Derimatol. Res. 293:430-433, 2001), suggesting exon 26A may confer a specific functional attribute involved in elastin function in keratinocytes.

Other exons of known functional significance are alternatively spliced with substantial frequency and in multiple tissues. For example, the glycine-rich hydrophobic domains contribute to the elasticity of the molecule. Skipping of these exons suggests that the tropoelastin produced has altered function. Among theses hydrophobic domains, those encoded by exons 2, 14, 28 and 30 are present in most or all tropoelastin isoforms. In contrast exons 3, 5, 7, 11, 32 and 33 are all subject to alternative splicing. Proximity may be important in some cases as exons 3, 5 and 7 are only individually skipped, yet exons 3 and 11, and 5 and II are sometimes skipped in combination.

The proline-rich hydrophobic domains are generally larger, and cluster in the center of the molecule. They also contribute to elasticity, but are seldom subject to alternative splicing. With the exception of exon 22, which is has not been confirmed in any human tropoelastin isoform, exon 9 encodes the only proline-rich hydrophobic domain known to be skipped. Splicing out of exon 9 is itself a low frequency event, and has been seen in only one transcript species. This particular transcript codes for an unusually short tropoelastin molecule, with 11 skipped exons.

The exons encoding crosslinking domains also show differences in alternative splicing. The most prevalent form of crosslinking domain in elastin, the lysine and alanine-rich crosslnking domains (KA domains), are largely constitutive. Of the ten KA crosslinking domains, only three of the corresponding exons are alternatively spliced. Exons 6, 19 and 23 are skipped albeit with relatively low frequency, while exons 15, 17, 21, 25, 27, 29 and 31 are included in all known transcripts. Skipping of exon 19 was unexpected as it had previously been identified as being involved in crosslink formation (Brown-Augsburger et al., J Biol Chem 270:17778-17783, 1995). Exons 10 and 19 are not coordinately expressed, indicating that each can find either an alternative crosslinking partner, or that they are always paired in crosslinks and both must be present in the transcript pool even if they are from different isoforms.

The lysine and proline-rich crosslinking domains (KP domains) show the opposite pattern to the I<A domains. There are five KP domains, encoded by exons 4, 8, 10, 12 and 13. Of these five, exons 8, 10 and 13 are all alternatively spliced. In particular, exon 13 is skipped in multiple transcript species, and surprisingly exons 8, 10 and 13 are all skipped in the same transcript. Taken together, these data indicate that KA domains are responsible for contributing crosslinking sites in all known tropoelastin isoforms regardless of splicing pattern, whereas KP domains appear to be less critical.

Exons that appear to be constitutive may confer particular functional or structural properties that are required, whereas redundancy in hydrophobic (elastogenic) and crosslinking domains allows for alternative use. For instance, exons responsible for activities such as cell binding (exons 2, 16, 24) (Senior et al., J. Cell Biol. 99:870-874, 1984; Wrenn et al, J. Biol. Chem. 262:2244-2249; Long et al., Biochim. Biophys. Acta 968:300-311, 1988; Robert, Connect. Tiss. Res. 40:75-82, 1999), interaction with elastin binding protein (exons 16, 24) (Mecham et al., Biochem. 28:3716-3722, 1989), elastic fiber assembly (exons 29, 30, 31 and 36) (Brown-Augsburger et al., J Biol Chem 270:17778-17783, 1995; Kozel et al., J Biol Chem 278:18491-18498, 2003; Hsiao et al, Connect Tiss. Res. 40:83-95, 1999; Rock et al., J. Biol. Chem. 279:23748-23758, 2004), coacervation (exon 26) (Jensen et al, J Biol Chem 275:28449-28454, 2000), and secretion from the cell (exon 1) (Bashir et al, J Biol Chem 264:8887-8891, 1989) are not alternatively spliced.

Three previously undescribed exons were identified in multiple transcripts isolated from the human fetal heart cDNA library (as described in additional detail in Example 1). A 15 bp sequence between exons 7 and 8 (exon 7A) adds a 5 amino acid residue sequence, APSVP (SEQ ID NO:15). An 18 nucleotide exon is present in some transcripts between exons 23 and 24 (exon 23A), which encodes the sequence ALLNLA (SEQ ID NO:16). Genomic analysis also reveals that there are alternatively spliced products that result from utilization of a cryptic donor splice site in exon 20. Use of the cryptic splice site maintains the reading frame but removes 26 amino acids. Consequently, what was previously defined as exon 20 is actually two distinct exons, 20 and 20A. If exon 20A is included, then there is a proline between domains 20A and 21. If exon 20A is skipped, then the junctional amino acid residue between domains 20 and 21 is an alanine.

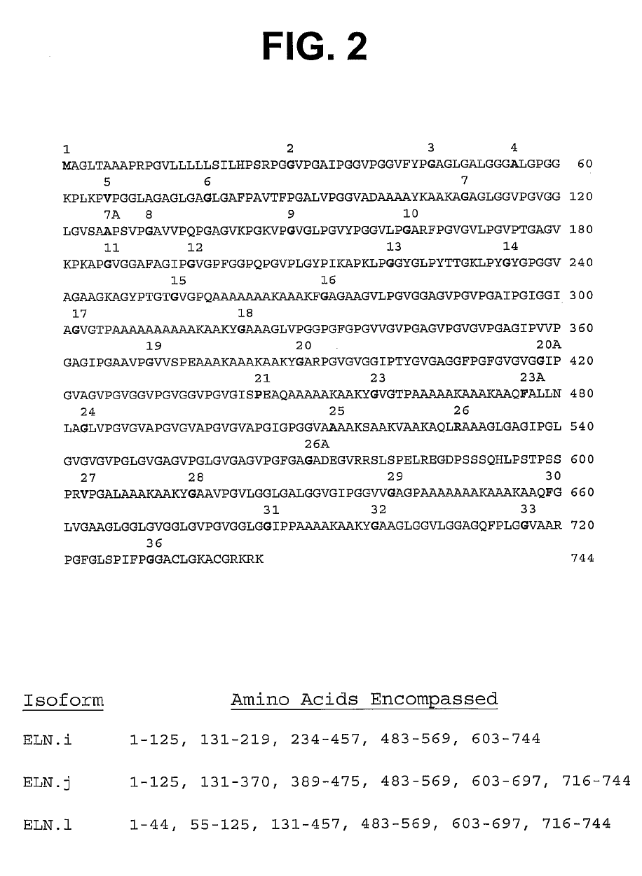

Analysis of 19 verified splice variants (enumerated in Table 2) indicated that, of the 37 ELN exons, 17 exons are subject to alternative splicing. Exons 3, 5, 6, 7, 7A, 8, 9, 10, 11, 13, 19, 20A, 23, 23A, 26A, 32 and 33 are all involved in exon skipping. Three alternatively spliced variants encoding three distinct tropoelastin isoforms were identified. Isoform I is encoded by a transcript that omits exons 7A, 13, 22, 23, 23A and 26A. Isoform J is encoded by a transcript that omits exons 7A, 19, 22, 23A, 26A, and 32. Isoform L is encoded by a transcript that omits exons 3, 7A, 22, 23, 23A, 26A, and 32. Exemplary polynucleotide sequences corresponding to tropoelastin isoform i, j and l nucleic acids are shown in SEQ ID NOs:1 and 7, 3 and 9, and 5 and 11, respectively. The two nucleotide sequences (e.g., 1 and 7) corresponding to a single tropoelastin isoform (i) represent polymorphic nucleic acids. An identical polymorphism is found in each isoform pair (that is, 1 and 7, 3 and 9, and 5 and 11), with the latter of each pair being the shorter polymorphic form. Exemplary amino acid sequences corresponding to isoforms I, J and L are shown in SEQ ID NOs:2 and 8, 4 and 10, and 6 and 12, respectively. As above, the paired amino acid sequences corresponding to a single isoform reflect a genomic polymorphism that results in the insertion of amino acids into the tropoelastin molecule at a position corresponding to between amino acids 503 and 504 of SEQ ID NO:14, which is a reference human tropoelastin including all of the translated exons. SEQ ID NOs:2, 4 and 6 represent the longer polymorphic forms of each isoform, that is, containing the amino acid insertion. The exon composition of isoforms I, J and L are illustrated in FIG. 2, with reference to the reference human tropoelastin molecule including all of the expressed exons (SEQ ID NO:14).

### Nucleic Acids Encoding Novel Tropoelastin Isoforms

Recombinant or isolated nucleic acids including polynucleotide sequences that encode these three previously undescribed tropoelastin isoforms (designated I, J and L) are features of this disclosure. Each of these polynucleotide sequences corresponds to a ELN transcript with a unique exon composition and includes a plurality of exons (and omits a plurality) of exons from among the 37 exons encoded by the ELN genomic sequence. The specific exon composition of each of these sequences has not previously been recognized in the art. In a first embodiment, the nucleic acids exclude (that is, skip or omit) exons 7A, 13, 22, 23, 23A and 26A (Isoform I). In a second embodiment, the nucleic acids exclude (that is, skip or omit) exons 7A, 19, 22, 23A, 26A, and 32 (Isoform J). In a third embodiment, the nucleic acids exclude (that is, skip or omit) exons 3, 7A, 22, 23, 23A, 26A (Isoform L).

For example, tropoelastin isoform i transcripts or cDNA (which encode tropoelastin I monomers) include exons 2, 3, 4, 5, 6, 7, 8, 9, 10, 11, 12, 14, 15, 16, 17, 18, 19, 20, 20A, 21, 24, 25, 26, 27, 28, 29, 30, 31, 32, 33, and 36, and omit exons 7A, 13, 22, 23, 23A and 26A of the human ELN genomic sequence. Tropoelastin isoform j transcripts or cDNA (which encode tropoelastin J monomers) include exons 2, 3, 4, 5, 6, 7, 8, 9, 10, 11, 12, 13, 14, 15, 16, 17, 18, 20, 20A, 21, 23, 24, 25, 26, 27, 28, 29, 30, 31, 33, and 36, and omit exons 7A, 19, 22, 23A, 26A, and 32 of the human ELN genomic sequence. Tropoelastin isoform 1 transcripts or cDNA (which encode tropoelastin L monomers) include exons 2, 4, 5, 6, 7, 8, 9, 10, 11, 12, 13, 14, 15, 16, 17, 18, 19, 20, 20A, 21, 24, 25, 26, 27, 28, 29, 30, 31, 33, and 36, and omit exons 3, 7A, 22, 23, 23A, 26A, and 32, of the human ELN genomic sequence. Optionally, each of these polynucleotide sequences can include exon 1 of the human ELN genomic sequence, which encodes a secretion signal sequence. These nucleic acids encode tropoelastin isoforms with distinct structural and/or functional properties, which are useful in the production of recombinant human tropoelastin, as well as elastin polymers suitable for use in elastin-based biomaterials.

Exemplary polynucleotide sequence encompassed by these embodiments are represented by NOs:1, 3 5, 7, 9 and 11. Exemplary amino acid sequences of tropoelastin isoform monomers are represented by SEQ ID NOs:2, 4 6, 8, 10 and 12. No published sequence or other reference has been identified that discloses the tropoelastin sequences encoded by these nucleic acids.

While each of SEQ ID NOs:1, 3 and 5 correspond to individual sequences isolated from a human fetal heart cDNA library, additional polynucleotide sequences that share the same exon composition but differ with respect to one or more nucleotides are equivalents within the context of this disclosure, e.g., SEQ ID NOs:7, 9 and 11.

Based on the human tropoelastin isoform proteins and corresponding nucleic acid sequences provided herein, one of ordinary skill in the art can easily produce numerous variants of these sequences using a variety of standard mutagenesis and cloning procedures. In one embodiment, variant tropoelastin proteins include proteins that differ in amino acid sequence from the human tropoelastin sequences disclosed but that share at least 80% amino acid sequence identity over substantially the entire length of a provided human tropoelastin protein. In other embodiments, other variants will share at least 85%, at least 90%, at least 95%, at least 98%, or at least 99% amino acid sequence identity over substantially the entire length of a tropoelastin isoform provided herein.

Sequence identity refers to the similarity between two amino acid sequences, or two nucleic acid sequences, and is frequently measured in terms of percentage identity (or similarity or homology); the higher the percentage, the more similar the two sequences are. Homologs (or other variants) of an amino acid or nucleic acid sequence will possess a relatively high degree of sequence identity when aligned using standard methods. Typically, variants are at least 70% similar (conserved) when compared at the amino acid level. A tropoelastin polypeptide or polynucleotide is identical (or for that matter, hybridizes) over substantially the entire length of a reference tropoelastin (such as any one SEQ ID NOs:1, 3, 5, 7, 9, or 11) if it is at least 80% identical on an amino acid for amino acid (or nucleotide for nucleotide) basis over the entire length of the reference sequence, and does not omit or include more than five contiguous amino acids (or 15 contiguous nucleotides) with respect to the reference sequence. For example, such a tropoelastin variant is at least 80%, or 85%, or 90% or 95%, or 98%, or 99% identical to SEQ ID NO:2 (or SEQ ID NO:4, or SEQ ID NO:6, or SEQ ID NO:8, or SEQ ID NO:10, or SEQ ID NO:12), and does not omit or include more that 5 contiguous amino acids with respect to the specified reference sequence. Similarly, a tropoelastin variant nucleic acid can be at least 80%, or 85%, or 90%, or 95% or 98% identical to, and does not omit or include more than 15 contiguous nucleotides as compared to one of SEQ ID NOs:1, 3, 5, 7, 9 or 11.

Methods of alignment of sequences for comparison are well known, and can readily be utilized to compare tropoelastin isoform proteins and their variants. Various programs and alignment algorithms are described in: Smith & Waterman, Adv. Appl. Matl. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al., Computer Appls. Biosci. 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and similarity/homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program.

Substantially similar sequences are typically characterized by possession of at least 80%, 85%, 90%, 95% or at least 98% sequence identity counted over substantially the entire length alignment with a sequence using the NCBI Blast 2.0, gapped blastp set to default parameters. Queries searched with the blastn program are filtered with DUST (Hancock and Armstrong, Comput. Appl. Biosci. 10:67-70, 1994). It will be appreciated that these sequence identity ranges are provided for guidance only; it is entirely possible that strongly variants could be obtained that fall outside of the ranges provided.

Tropoelastin isoform variants can be produced by manipulation of the nucleotide sequence encoding tropoelastin using standard procedures. For instance, in one specific, non-limiting, embodiment, site-directed mutagenesis or in another specific, non-limiting, embodiment, PCR, can be used to produce such variants. The simplest modifications involve the substitution of one or more amino acids for amino acids having similar biochemical properties. These so-called conservative substitutions are likely to have minimal impact on the activity of the resultant protein. Table 1 provides a summary of conservative amino acid substitutions based on a BLOSUM similarity matrix.

TABLE 1
Conservative Amino Acid Substitutions

| Amino Acid | Conservative Substitutions |
|---|---|
| G | A, S, N |
| P | E |
| D | S, K, Q, H, N, E |
| E | P, D, S, R, K, Q, H, N |
| N | G, D, E, T, S, R, K, Q, H |
| H | D, E, N, M, R, Q |
| Q | D, E, N, H, M, S, R, K |
| K | D, E, N, Q, R |
| R | E, N, H, Q, K |
| S | G, D, E, N, Q, A, T |
| T | N, S, V, A |
| A | G, S, T, V |
| M | H, Q, Y, F, L, I, V |
| V | T, A, M, F, L, I |
| I | M, V, Y, F, L |
| L | M, V, I, Y, F |
| F | M, V, I, L, W, Y |
| Y | H, M, I, L, F, W |
| W | F, Y |
| C | None |

In another embodiment, more substantial changes in tropoelastin function can be obtained by selecting amino acid substitutions that are less conservative than the conservative substitutions listed in Table 1. In one specific, non-limiting, embodiment, such changes include changing residues that differ more significantly in their effect on maintaining polypeptide backbone structure (e.g., sheet or helical conformation) near the substitution, charge or hydrophobicity of the molecule at the target site, or bulk of a specific side chain. The following specific, non-limiting, examples are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histadyl) is substituted for (or by) an electronegative residue (e.g., glutamyl or aspartyl); or (d) a residue having a bulky side chain (e.g., phenylalanine) is substituted for (or by) one lacking a side chain (e.g., glycine).

In other embodiments, changes in tropoelastin structure and/or function can be obtained by mutating, substituting or deleting regions of tropoelastin that have a known function, regions where the function is yet to be determined, or regions that are known to be highly conserved or not conserved, without substantially altering the exon structure of the tropoelastin isoform. For example, without substantially altering the exon structure of a tropoelastin isoform, amino acid substitutions (or deletions or additions) can be made that add or remove a cross-linking site, or that add or remove a cell-binding site. Such modifications are predicted to alter the structural and/or functional properties of the tropoelastin isoform.

Variant tropoelastin encoding sequences can be produced by standard DNA mutagenesis techniques. In one specific, non-limiting, embodiment, M13 primer mutagenesis is performed. Details of these techniques are provided in Sambrook et al. (In Molecular Cloning: A Laboratoiy Manual, CSHL, New York, 1989), Ch. 15. By the use of such techniques, variants can be created that differ in minor ways from the human tropoelastin sequences disclosed. In one embodiment, nucleic acids and polynucleotide sequences that are derivatives of those specifically disclosed herein, and which differ from those disclosed by the deletion, addition, or substitution of nucleotides while still encoding a protein that has at least 80% or greater sequence identity (such as 85%, 90%, 95%, or 98% sequence identity) to one or more of human tropoelastin isoforms I, J and L are comprehended by this disclosure. For example, closely related nucleic acid molecules that share at least 75%, at least 80%, at least 85%, at least 90%, at least 95%, or at least 98% nucleotide sequence identity with the disclosed tropoelastin encoding sequences are comprehended by this disclosure. In certain embodiments, related nucleic acid molecules encode tropoelastin proteins with no more than 1, 3, 5, or 10 amino acid changes compared to one of SEQ ID NO:2, SEQ ID NO:4, SEQ ID NO:6, SEQ ID NO:8, SEQ ID NO:10 and/or SEQ ID NO:12.

In some embodiments, the coding region can be altered by taking advantage of the degeneracy of the genetic code to alter the coding sequence such that, while the nucleotide sequence is substantially altered, it nevertheless encodes a protein having an amino acid sequence substantially similar to the disclosed human tropoelastin protein sequences. For example, because of the degeneracy of the genetic code, four nucleotide codon triplets—(GCT, GCG, GCC and GCA)—code for alanine. The coding sequence of any specific alanine residue within the human tropoelastin protein, therefore, could be changed to any of these alternative codons without affecting the amino acid composition or characteristics of the encoded protein. Based upon the degeneracy of the genetic code, variant DNA molecules can be derived from the cDNA and gene sequences disclosed herein using standard DNA mutagenesis techniques as described above, or by synthesis of DNA sequences. Thus, this disclosure also encompasses nucleic acid sequences that encode a tropoelastin protein, but which vary from the disclosed nucleic acid sequences by virtue of the degeneracy of the genetic code.

As will be recognized by one of ordinary skill in the art, numerous such variants can be made by substituting a nucleotide, e.g., in the third position of a codon, without altering the polypeptide encoded by the nucleic acid.

For example, it is often desirable to alter the polynucleotide sequence for the purpose of improving expression in a particular expression host. The tropoelastin nucleic acids disclosed herein correspond to human ELN transcripts, and as such encode a polypeptide in accordance with the codon preference found in mammalian (e.g., human cells). For expression in bacterial cells, it is often desirable to substitute one or more nucleotides so that the polypeptide is encoded by codons that are consistent with the codon usage in the bacterial cells. Similarly, if expression is desired in plant cells, it can be useful to substitute one or more nucleotides so that the polypeptide is encoded by codons that are consistent with the codon usage in plant cells. Substituting nucleotides to alter the codon composition to conform to that of a selected expression host is referred to as “codon optimization” and the resulting nucleic acid is said to be “codon optimized” with respect to the selected expression host. Nucleic acid variants of SEQ ID NOs:1, 3, 5, 7, 9 and/or 11 that are codon optimized for expression in host cells (such as bacterial host cells) are expressly contemplated by this disclosure. U.S. Pat. No. 6,232,458 and Martin et al., Gene 154:159-166, 1995, which are incorporated herein by reference, provide a detailed discussion of the design and production of codon optimized tropoelastin isoforms, and are sufficient to guide one of ordinary skill in the art in the production of codon optimized nucleic acids that encode isoforms I, J and L (represented by SEQ ID NOs:2 and 8, 4 and 10, and 6 and 12, respectively).

In one embodiment, such variants can differ from the disclosed sequences by alteration of the coding region to fit the codon usage bias of the particular organism into which the molecule is to be introduced.

Nucleic acid molecules that are derived from the human tropoelastin cDNA nucleic acid sequences include molecules that specifically hybridize to the disclosed prototypical tropoelastin (ELN) cDNA molecules, and fragments thereof. Specific hybridization refers to the binding, duplexing, or hybridizing of a molecule only or substantially only to a particular nucleotide sequence (such SEQ ID NO:1, SEQ ID NO:3, SEQ ID NO:5, SEQ ID NO:7, SEQ ID NO:9 and/or SEQ ID NO:11) when that sequence is present in a complex mixture (e.g., total cellular DNA or RNA). Specific hybridization can occur under conditions of varying stringency.

Hybridization conditions resulting in particular degrees of stringency will vary depending upon the nature of the hybridization method of choice and the composition and length of the hybridizing DNA used. Generally, the temperature of hybridization and the ionic strength (especially the Na⁺ concentration) of the hybridization buffer will determine the stringency of hybridization. Calculations regarding hybridization conditions required for attaining particular degrees of stringency are discussed by Sambrook et al (In: Molecular Cloning: A Laboratory Manual, Cold Spring Harbor, N.Y., 1989 ch. 9 and 11). By way of illustration only, a hybridization experiment can be performed by hybridization of a DNA molecule to a target DNA molecule which has been electrophoresed in an agarose gel and transferred to a nitrocellulose membrane by Southern blotting (Southern, J. Mol. Biol. 98:503, 1975), a technique well known in the art and described in Sambrook et al. (Molecular Cloning: A Laboratory Manual, Cold Spring Harbor, N.Y., 1989).

Traditional hybridization with a target nucleic acid molecule labeled with [³²P]-dCTP is generally carried out in a solution of high ionic strength such as 6×SSC at a temperature that is 20-25° C. below the melting temperature, Tₘ, described below. For Southern hybridization experiments where the target DNA molecule on the Southern blot contains 10 ng of DNA or more, hybridization is typically carried out for 6-8 hours using 1-2 ng/ml radiolabeled probe (of specific activity equal to 10⁹ CPM/μg or greater). Following hybridization, the nitrocellulose filter is washed to remove background hybridization. The washing conditions should be as stringent as possible to remove background hybridization but to retain a specific hybridization signal.

The term Tₘ represents the temperature (under defined ionic strength, pH and nucleic acid concentration) at which 50% of the probes complementary to the target sequence hybridize to the target sequence at equilibrium. Because the target sequences are generally present in excess, at Tₘ 50% of the probes are occupied at equilibrium. The Tₘ of such a hybrid molecule can be estimated from the following equation (Bolton and McCarthy, Proc. Natl. Acad. Sci. USA 48:1390, 1962):

Tₘ=81.5° C.-16.6(log₁₀[Na⁺])+0.41(% G+C)−0.63(% formamide)−(600/l)

where 1=the length of the hybrid in base pairs.

This equation is valid for concentrations of Na⁺ in the range of 0.01 M to 0.4 M, and it is less accurate for calculations of Tm in solutions of higher [Na⁺]. The equation is also primarily valid for DNAs whose G+C content is in the range of 30% to 75%, and it applies to hybrids greater than 100 nucleotides in length (the behavior of oligonucleotide probes is described in detail in Ch. 11 of Sambrook et al. (Molecular Cloning: A Laboratory Manual, Cold Spring Harbor, N.Y., 1989).

Thus, by way of example, for a 150 base pair DNA probe derived from a nucleic acid that encodes tropoelastin (e.g., with a hypothetical % GC of 45%), a calculation of hybridization conditions required to give particular stringencies can be made as follows: For this example, it is assumed that the filter will be washed in 0.3×SSC solution following hybridization, thereby: [Na⁺]=0.045 M; % GC=45%; Formamide concentration=0; 1=150 base pairs; Tₘ=81.5−16.6(log₁₀[Na+])+(0.41×45)−(600/150); and so Tm=74.4° C.

The Tₘ of double-stranded DNA decreases by 1-1.5° C. with every 1% decrease in homology (Bonner et al., J. Mol. Biol. 81:123, 1973). Therefore, for this given example, washing the filter in 0.3×SSC at 59.4-64.4° C. will produce a stringency of hybridization equivalent to 90%; that is, DNA molecules with more than 10% sequence variation relative to the target cDNA will not hybridize. Alternatively, washing the hybridized filter in 0.3×SSC at a temperature of 65.4-68.4° C. will yield a hybridization stringency of 94%; that is, DNA molecules with more than 6% sequence variation relative to the target cDNA molecule will not hybridize. The above example is given entirely by way of theoretical illustration. It will be appreciated that other hybridization techniques can be utilized and that variations in experimental conditions will necessitate alternative calculations for stringency.

Stringent conditions can be defined as those under which DNA molecules with more than 25%, 15%, 10%, 6% or 2% sequence variation (also termed “mismatch”) will not hybridize. Stringent conditions are sequence dependent and are different in different circumstances. Longer sequences hybridize specifically at higher temperatures. Generally, stringent conditions are selected to be about 5° C. lower than the thermal melting point Tₘ for the specific sequence at a defined ionic strength and pH. An example of stringent conditions is a salt concentration of at least about 0.01 to 1.0 M Na ion concentration (or other salts) at pH 7.0 to 8.3 and a temperature of at least about 30° C. for short probes (e.g., 10 to 50 nucleotides). Stringent conditions can also be achieved with the addition of destabilizing agents such as formamide. For example, conditions of 5× SSPE (750 mM NaCl, 50 mM Na Phosphate, 5 mM EDTA, pH 7.4) and a temperature of 25-30° C. are suitable for allele-specific probe hybridizations.

A perfectly matched probe has a sequence perfectly complementary to a particular target sequence. The test probe is typically perfectly complementary to a portion (subsequence) of the target sequence. The term “mismatch probe” refers to probes whose sequence is selected not to be perfectly complementary to a particular target sequence.

With the provision herein of the sequences of specific tropoelastin isoforms (SEQ ID NOs:2, 4, 6, 8, and 12) and cDNAs encoding them (SEQ ID NOs:1, 3, 5, 7, 9 and 11, and variants thereof), in vitro nucleic acid amplification (such as polymerase chain reaction (PCR)) can be utilized as a simple method for producing tropoelastin encoding sequences. The following provides representative techniques for preparing cDNA in this manner.

Total RNA is extracted from human cells by any one of a variety of methods well known to those of ordinary skill in the art. Sambrook et al. (In Molecular Cloning: A Laboratory Manual, CSHL, New York, 1989) and Ausubel et al. (In Current Protocols in Molecular Biology, Greene Publ. Assoc. and Wiley-Intersciences, 1992) provide descriptions of methods for RNA isolation. Tropoelastin is expressed in many different human tissues and cell lines. In one embodiment, primary cells are obtained from normal tissues, such as fetal heart. In yet another embodiment cell lines, derived from normal or neoplastic tissues, are used as a source of such RNA. The extracted RNA is then used, for example as a template for performing reverse transcription (RT)-PCR amplification of cDNA. Methods and conditions for RT-PCR are described in Kawasaki et al., (In PCR Protocols, A Guide to Methods and Applications, Innis et al. (eds.), 21-27, Academic Press, Inc., San Diego, Calif., 1990).

The selection of amplification primers will be made according to the portion(s) of the cDNA that is to be amplified. In one embodiment, primers can be chosen to amplify a segment of a cDNA or, in another embodiment, the entire cDNA molecule. Variations in amplification conditions can be required to accommodate primers and amplicons of differing lengths and composition; such considerations are well known in the art and are discussed for instance in Innis et al. (PCR Protocols, A Guide to Methods and Applications, Academic Press, Inc., San Diego, Calif., 1990). One skilled in the art will appreciate that many different primers can be derived from the provided cDNA sequence in order to amplify particular regions of a tropoelastin encoding cDNA, as well as the complete sequence of any human tropoelastin encoding cDNA.

Re-sequencing of PCR products obtained by amplification procedures optionally can be performed to facilitate confirmation of the amplified sequence and provide information about natural variation of this sequence in different populations or species. Oligonucleotides derived from the provided tropoelastin sequences can be used in such sequencing methods.

Unique oligonucleotides corresponding to sequences derived from previously undescribed exons of the human tropoelastin cDNA sequence (e.g., SEQ ID NO:1, SEQ ID NO:3, SEQ ID NO:5, SEQ ID NO:7, SEQ ID NO:9 or SEQ ID NO:11) are also encompassed within the scope of the present disclosure. In one embodiment, such oligonucleotides can include a sequence of at least 10 consecutive nucleotides of the unique tropoelastin exon sequence. If these oligonucleotides are used with an in vitro amplification procedure (such as PCR), lengthening the oligonucleotides can enhance amplification specificity. Thus, in other embodiments, oligonucleotide primers comprising at least 15, 20, 25, 30, 35, 40, 45, 50, or more consecutive nucleotides (including all or a portion of a unique and previously undescribed exon of these sequences can be used.

Human tropoelastin isoform encoding nucleic acid molecules (including the cDNAs, amplification products, and the like including the polynucleotide sequences represented by SEQ ID NO:1, 3, 5, 7, 9, and/or 11, and variants of these sequences) can be incorporated into transformation or expression vectors, such as plasmids.

Vector systems suitable for the expression of tropoelastin isoforms include the pUC series of vectors (Vieira and Messing, Gene 19:259-268, 1982) pUR series of vectors (Ruther and Muller-Hill, EMBO J. 2:1791, 1983), pEX1-3 (Stanley and Luzio, EMBO J. 3:1429, 1984) and pMR100 (Gray et al, Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET vectors (Studiar and Moffatt, J. Mol. Biol. 189:113, 1986) and pGEX vectors (GE Healthcare, formerly Amersham Biosciences).

The DNA sequences can also be transferred from their existing context to other cloning vehicles, such as other plasmids, bacteriophages, cosmids, animal viruses and yeast artificial chromosomes (YACs) (Burke et al., Science 236:806-812, 1987). These vectors can then be introduced into a variety of hosts including somatic cells, and simple or complex organisms, such as bacteria, fingi (Timberlake and Marshall, Science 244:1313-1317, 1989), invertebrates, plants (Gasser and Fraley, Science 244:1293, 1989), and animals (Hogan et al., Manipulating the Mouse Embryo: A Laboratory Manual, (2^{nd} ed.) Cold Spring Harbor Laboratory Press, Cold Spring Harbor, 1994; Pursel et al., Science 244:1281-1288, 1989), which cell or organisms are rendered transgenic by the introduction of the heterologous cDNA.

For expression in mammalian cells, a tropoelastin encoding cDNA sequence can be ligated to a heterologous promoter, such as the simian virus (SV) 40 promoter in the pSV2 vector (Mulligan and Berg, Proc. Natl. Acad. Sci. USA 78:2072-2076, 1981), and introduced into mammalian cells, such as monkey COS-1 cells (Gluzman, Cell 23:175-182, 1981), to achieve transient or long-term expression. The tropoelastin encoding nucleic acids can also be introduced into cells, such as stem cells and reintroduced into a subject to achieve expression of the a tropoelastin isoform in vivo. Alternatively, the tropoelastin cDNA sequence can be operably linked to transcription control sequences that are active (either constitutively or inducibly) in bacterial (such as E. coli) cells.

A tropoelastin cDNA (for instance, a codon optimized tropoelastin cDNA) can be ligated into bacterial expression vectors by conventional techniques, and introduced into a suitable bacterial expression host. Alternatively, the cDNA sequence (or portions derived from it) or a mini gene (a cDNA with an intron and its own promoter) can be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci. USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985)) or by using vectors that contain promoters amenable to modulation, for example, the glucocorticoid-responsive promoter from the mouse mammary tumor virus (Lee et al, Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or Leo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomai, freely replicating entities by using regulatory elements of viruses such as papilloma (Sarver et al., Mol. Cell. Biol. 1:486, 1981) or Epstein-Barr (Sugden et al., Mol. Cell. Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

Similarly, the transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology 52:466, 1973) or strontium phosphate (Brash et al., Mol. Cell. Biol. 7:2013, 1987), electroporation (Neumann et al., EMBO J. 1:841, 1982), lipofection (Feigner et al., Proc. Natl. Acad. Sci. USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors. Systems are developed that use, for example, retroviruses (Bernstein et al., Gen. Engrag 7:235, 1985), adenoviruses (Ahmad et al., J. Virol. 57:267, 1986), or Herpes virus (Spaete et al., Cell 30:295, 1982). Additionally, nucleic acids encoding tropoelastin isoforms can also be delivered to target cells in vitro via non-infectious systems, for instance liposomes.

### Expression of Tropoelastin Isoform Proteins

Using nucleic acids that encode tropoelastin isoforms, such as the exemplary nucleotide sequences shown in SEQ ID NOs:1, 3, 5, 7, 9 and 11, or variants, such as codon optimized versions, thereof, one of ordinary skill in the art can produce (e.g., express and purify) recombinant tropoelastin isoforms using standard laboratory techniques. Purified human tropoelastin monomers, as well as elastin polymers produced therefrom, can be used for functional analyses, antibody production, and biomaterial production (e.g., for patient therapy).

For example, the DNA sequence of the tropoelastin isoform cDNA (e.g., SEQ ID NO:1, 3, 5, 7, 9, and/or 11) can be manipulated in studies to understand the expression of the gene and the function of its product, such as by altering a structural feature or domain of the protein. In other embodiments, mutant forms of the human tropoelastin isoforms can be isolated based upon information contained herein, and can be studied in order to detect alteration in expression patterns in terms of relative quantities, cellular localization, tissue specificity and functional properties of the encoded mutant tropoelastin isoform proteins.

Methods for expressing large amounts of protein from a cloned gene introduced into Escherichia coli (E. coli) can be utilized for the purification of proteins. For example, nucleic acids encoding fusion proteins that include an amino terminal domain with a portion of the glutathione s-transferase (GST) protein linked to all or a portion of a tropoelastin isoform can be used to prepare proteins, including tropoelastin monomers. The recombinant elastin monomers are useful, for example, for producing synthetic elastin polymers or other biomaterials that include elastin. Alternatively, the purified monomers can be used to produce polyclonal or monoclonal antibodies. For example, by expressing the novel isoforms described herein, polyclonal and/or monoclonal antibodies can be produced that specifically bind to unique epitopes not found in other tropoelastin isoforms. Thereafter, these antibodies can be used for a variety of purposes, including purification of proteins by immunoaffinity chromatography, in diagnostic assays to quantitate the levels of protein and to localize proteins in tissues and individual cells by immunofluorescence.

Methods and plasmid vectors for producing tropoelastin fusion proteins and intact native proteins in culture are well known in the art, and specific methods are described in Suitable methods are described in U.S. Pat. Nos. 6,232,458 and 7,001,328, and Martin et al., Gene, 154:159-166, which are incorporated herein by reference. Additional details regarding production of recombinant vectors useful in the context of producing recombinant expression vectors that encode tropoelastin can be found in Sambrook et al. (In Molecular Cloning: A Laboratory Manual, Ch. 17, CSHL, New York, 1989). Such proteins can be made in large amounts, are easy to purify, and can be used, for instance for functional assays or as the starting material for the production of elastin polymers and biomaterials. Proteins can be produced in bacteria by placing a strong, regulated promoter and an efficient ribosome-binding site upstream of the cloned gene. If low levels of protein are produced, additional steps can be taken to increase protein production; if high levels of protein are produced, purification is relatively easy. Suitable methods are presented in Sambrook et al. (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning: A Laboratory Manual, Ch. 17, CSHL, New York, 1989).

Using the above techniques, expression vectors containing a tropoelastin (or tropoelastin fusion protein) encoding sequence or cDNA, or fragments or variants or mutants thereof, can be introduced into suitable bacterial or mammalian cells. The choice of cell is influenced by the purpose for which the tropoelastin isoform protein is being produced. For example, production in bacterial cells permits the recovery of large amounts of tropoelastin monomer suitable for coacervation, cross-linking to produce synthetic elastin polymers and elastin-based biomaterials.

The present disclosure thus encompasses recombinant vectors that comprise all or part of a tropoelastin isoform nucleic acid (such as SEQ ID NOS:1, 3, 5, 7, 9 and 11, and variants thereof, e.g., variants that have been codon optimized for expression in a selected host cell), for expression in a suitable host, either alone or as a fusion protein, such as a labeled or otherwise detectable or readily isolatable fusion protein. The DNA is operably linked (that is, placed under the transcription regulatory control) in the vector to an expression control sequence so that a tropoelastins monomer polypeptide or tropoelastin fusion (e.g., GST-tropoelastin fusion) polypeptide can be expressed. Numerous commercially available expression vectors are available, that optionally include a polynucleotide sequence that encodes a GST domain (e.g. pET series vectors, pGEX series vectors, and the like). Optionally, a polynucleotide sequence that encodes a portion of a tropoelastin monomer that lacks the amino acids encoded by exon 1 is utilized, for example, to improve recovery in bacterial expression systems.

The expression control sequence can be selected from the group consisting of sequences that control the expression of genes of prokaryotic or eukaryotic cells, their viruses, and combinations thereof. The expression control sequence can be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which can be transfected with the vector of this disclosure, can be selected from the group consisting of E. coli, Pseudomonas, Bacillus subtilis, Bacillus stearothermophilus or other bacilli; other bacteria; yeast; fungi; insect (such as S. frugiperda); mouse or other animal; plant hosts; or human tissue cells.

It is appreciated that for mutant or variant tropoelastin (ELN) sequences, similar systems are employed to express and produce the mutant product. In addition, fragments of a tropoelastin protein (such as fragments lacking sequences encoded by exon 1) can be expressed essentially as detailed above, as can fusion proteins comprising all of tropoelastin or a fragment or fragments thereof fused to a polypeptide other than tropoelastin. Such fragments include individual tropoelastin protein domains or sub-domains, as well as shorter fragments such as peptides.

Production of large quantities of tropoelastin can be performed by scaling up the procedures disclosed herein, for example by growing multiple vessels of host cells or by adapting the procedures outlined herein (or in references incorporated herein) for growth of the selected host cell in a large scale culture vessel, such as a 10 liter or 100 liter bioreactor. Such scale up is within ordinary skill.

In some embodiments, it is beneficial to obtain isolated and purified tropoelastin monomers, for example. in the production of synthetic elastin polymers and/or elastin-based biomaterials. One skilled in the art will understand that there are myriad ways to purify recombinant polypeptides, and such typical methods of protein purification can be used to purify the disclosed tropoelastin monomer fusion proteins. Such methods include, for instance, protein chromatographic methods including ion exchange, gel filtration, HPLC, monoclonal antibody affinity chromatography and isolation of insoluble protein inclusion bodies after over production. In addition, purification affinity-tags, for example, GST or a six-histidine sequence, can be recombinantly fused to the protein and used to facilitate polypeptide purification (e.g., in addition to another functionalizing portion of the fusion, such as a targeting domain or another tag, or a fluorescent protein, peptide, or other marker). A specific proteolytic site, for instance a thrombin-specific digestion site, can be engineered into the protein between the tag and the remainder of the fusion to facilitate removal of the tag after purification, if such removal is desired.

Commercially produced protein expression/purification kits provide tailored protocols for the purification of proteins made using each system. See, for instance, the QIAexpress™ expression system from QIAGEN (Chatsworth, Calif.) and various expression systems provided by INVITROGEN (Carlsbad, Calif.). Where a commercial kit is employed to produce an tropoelastin monomer, the manufacturer's purification protocol is a preferred protocol for purification of that protein. For example, tropoelastin expressed as a GST fusion can be purified using a glutathione conjugated substrate such as a glutathione agarose matrix; proteins expressed with an amino-terminal hexa-histidine tag can be purified by binding to nickel-nitrilotriacetic acid (Ni-NTA) metal affinity chromatography matrix (The QIAexpressionist, QIAGEN, 1997).

In addition to protein expression and purification guidelines provided herein, protein expression/purification kits are produced commercially. See, for instance, the QIAexpress™ expression system from QIAGEN (Chatsworth, Calif.) and various expression systems provided by INVITROGEN (Carlsbad, Calif.). Suitable protocols for the production and purification of tropoelastin monomers can be selected or adapted from the instruction provided with these kits.

Exemplary and non-limiting procedures for purifying recombinant tropoelastin monomers are described in Wu and Weiss, Eur. J. Biochem., 266:308-314 and Bedell-Hogan et al., J. Biol. Chem., 268:10345-10350, which are incorporated herein by reference. In brief, a crude preparation can be purified using one or more preparative columns, such as cation exchange (e.g., BioRad HS50, SP Sepharose) and/or reversed phase columns. In one example, the tropoelastin monomers are applied to a reversed phase column (e.g., Vydac C4 21×25 mm) and eluted at room temperature with an acetonitrile gradient (0-80%). The recovered tropoelastin-containing eluate is desalted by dialyzing against 0.1% trifluoroacetic acid and if desired lyophilized for storage. Optionally, one or more tropoelastin-containing fractions can be pooled to reduce processing steps.

### Production of Synthetic Elastin Polymers

Elastin-based biomaterials are being developed as a biocompatible material used in tissue repair or replacement, as well as other biomaterials, due to its biological inertness and mechanical resilience. Because the biological and mechanical properties of biomaterials are influenced by elastin isoform composition, successful design and deployment of an elastin based biomaterials will require appropriate isoforms to be used according to biomaterial location and intended function.

Recombinant tropoelastin monomers (including isoform I, J and L monomers) are favorably used in the production of synthetic elastin polymers and other elastin-based biomaterials. The use of recombinant tropoelastin makes it possible to modify not only the physical properties of the biomaterial, but the manner and degree to which cells respond to the elastin biomaterial. For example, cell-binding sites or protein-interaction sites can be added or replaced to produce elastin based biomaterials suitable for specific applications and to optimize performance in vivo. For example, implanted natural elastin biomaterials tend to calcify via a poorly understood mechanism. Calcification of vascular tissue is also a common finding, for example in prosthetic valve replacements, as well as in atherosclerosis, diabetes, renal failure, ageing, and aortic stenosis (Wallin et al., Medical Research Reviews 21:274-301, 2001). Thus, by appropriately engineering the nucleic acids encoding the tropoelastin, the propensity of the elastin biomaterial to calcify can be minimized. Thus, using the methods disclosed herein to produce recombinant tropoelastin offers the significant advantage of being able to add or remove sequences to tailor biological properties, as well as increasing production and reducing cost of producing elastin-based biomaterials.

Accordingly, recombinant tropoelastin monomers (such as tropoelastin isoforms I, J and L) can be produced as described above, and then coacervated and cross-linked to produce synthetic elastin polymers and elastin-based biomaterials. The individual isoforms can be utilized singly or in any combination to form elastin polymers of differing composition. Thus, a synthetic elastin polymer can be produced that includes a single tropoelastin isoform selected from isoform I, isoform J or isoform L. Alternatively, a synthetic elastin polymer can be produced that includes one, two or even all three of isoforms I, J and L. Optionally, the synthetic isoform can also include one or more previously described tropoelastin isoforms, such as isoform A (or any other tropoelastin isoform or combination thereof with desirable properties, such another tropoelastin isoform designated in Table 2).

For example, a synthetic elastin polymer and/or elastin based biomaterial can be produced by introducing a recombinant nucleic acid including a polynucleotide sequence that encodes the selected tropoelastin isoform monomer as a GST fusion protein. For example, any one or more of SEQ ID NOs:1, 3, 5, 7, 9, and 11 can be used for this purpose. Alternatively, a variant of such a sequence (for instance, that has been codon optimized for expression in a host cell) can favorably be used to produce recombinant tropoelastin monomer. By way of example, such nucleic acid can be ligated into a pGEX (e.g., pGEX-2T) vector in frame with the GST fusion partner and operably linked to the IPTG inducible tac promoter. Following production of the GST-tropoelastin fusion protein in E. coli (or another host cell), the tropoelastin monomer is liberated from the GST moiety by cyanogens bromide or enzymatic cleavage, and purified (e.g., by column chromatography). Alternatively, a polynucleotide sequence encoding the desired tropoelastin isoform lacking exon 1 is ligated downstream of an ATG site operably linked to an inducible promoter and directly expressed (that is, without a fusion partner).

Following expression and purificafion (e.g., as described herein), a solution of one or more tropoelastin isoforms in physiological buffer is prepared at room temperature, sterile filtered, and optionally warmed to 37° C. The solution is incubated for a time sufficient for coacervate formation (e.g., until maximum turbidity is attained). The coacervate forms oily droplets, which can be harvested by centrifugation. After centrifugation, the tropoelastin remains in the lower phase. Optionally, the supernatant (upper phase) can be concentrated and the procedure repeated to increase the recovery of tropoelastin. One gram of recombinant tropoelastin produces approximately one ml of coacervate, which can be used to prepare a cross-linked polymer.

Tropoelastin coacervates can be cross-linked using naturally occurring or synthetic cross-linking agents, as described in U.S. Pat. No. 6,232,458. So long as the natural cross-link initiator lysyl oxidase is not available commercially in large quantities, such chemical cross-linkers are typically preferable for applications requiring large amounts of synthetic elastin polymer. For example, a bi-functional chemical cross-linker, such as bis(sulfosuccinimidyl)suberate (BSS), can favorably be used to cross-link recombinant tropoelastin coacervates. BSS introduces a (CH₂)₆— between two lysine residues. This is a minor modification that does not appreciably affect the structural or functional properties of the resulting synthetic elastin polymer.

Cross-linking of a tropoelastin coacervate can be performed at a range of temperatures, for example, between about −20° C. to about 37° C., with the rate of polymerization being increased at increased temperatures. For example, cross-linking can be performed by prechilling a coacervate to approximately −20° C. The cross-linking agent (such as BSS, Pierce) is stirred into the sample to assure complete mixing. The solution can then be poured into a pre-chilled cast of the desired dimension. The cast is then warmed to 37° C. for 48 hours to complete cross-linking. Additional methods for producing synthetic elastin polymers (including elastin hydrogels) are described in Mithieux et al, Biomaterials 25:4921-4927, 2004.

Synthetic elastin polymers that include isoforms I, J and/or isoform L, singly or in combination with each other or another tropoelastin isoform, can be produced using analogous methods by expressing a nucleic acid encoding the selected isoform. Elastin polymers comprising combinations of isoforms I, J and/or L (and/or additional tropoelastin isoforms) can be produced by combining the various recombinant tropoelastin isoforms in the desired ratio (for example, prior to coacervation and in vitro cross-linking). Thus, elastin polymers comprising isoforms I, J and/or L in combination, optionally in combination with one or more additional representative isoforms, can be produced that possess desired functional attributes, e.g., with respect to strength, elasticity, cell attachment, and the like.

### Uses of Novel Elastin and Tropoelastins

Properties of elastin make it a useful material from which to fabricate biomaterials. It is elastic, durable, and resists degradation, has a low immunogenicity and is well tolerated by most subjects (both human and non-human).

Elastin and elastin-based biomaterials, or tropoelastin materials incorporating isoforms I, J and L, can be used in a number of medical applications, such as those described in U.S. Pat. Nos. 6,372,228, 6,632,450, and 6,667,051, which are incorporated herein by reference. For example, these materials can be employed to provide a method of effecting repair or replacement or supporting a section of a body tissue, as a stent (such as a vascular stent), or as conduit replacement, or as an artery, vein or a ureter replacement, or as a stent or conduit covering or coating or lining. It can also provide a graft suitable for use in repairing a lumen wall, or in tissue replacement or repair in, for example, interior bladder replacement or repair, intestine, tube replacement or repair such as fallopian tubes, esophagus such as for esophageal varicies, ureter, artery such as for aneurysm, vein, stomach, lung, heart such as congenital cardiac repair, or colon repair or replacement, or skin repair or replacement, or as a cosmetic implant or breast implant. Further exemplary applications and methods for utilizing the tropoelastin isoforms and elastin polymers produced using the tropoelastin isoforms are disclosed, e.g., in U.S. Pat. Nos. 5,989,244 5,990,379, 6,632,450, 6,372,228, 6,667,051, and 7,001,328, which are incorporated herein by reference.

### Differential Isoform Properties

The elastic properties of different tissues is dictated, at least to some degree, by the nature of the elastic fibers specific to that tissue. The tropoelastin isoform composition of the elastic fibers is a basis for these differences in elastic properties. Consequently, understanding alternative splicing of ELN and the transcriptional combinations produced by different tissues is important for developing elastin as a component of biomaterials, as well as for determining the role of elastin in tissue homeostasis and disease.

The identification of nucleic acids that encode novel tropoelastin isoforms, and the recognition of differences in biological properties between the different isoforms makes it possible to design recombinant human tropoelastin molecule(s) with altered, tailored, and/or optimized properties for a biomaterial, for instance as defined by in vitro tests, such as those described herein. It is also possible to form biomaterials for specific applications, which can be tested in vivo using animals (e.g., transgenic animals expressing one or more human tropoelastin isoforms).

For example, there is variation in the strength of elastin patches, cell attachment properties and calcifying tendencies of the various tropoelastin isoforms. Based on the teachings disclosed herein, it is possible to design an isoform (or several) that has several advantageous properties combined into one molecule, for example excellent mechanical strength with good cell attachment properties and minimum calcification. By way of example, such engineered “isoforms” of tropoelastin can be generated by switching out or selecting exons that are characterized as conveying or influencing a property of the resultant elastin. One or more of the novel tropoelastin isoforms disclosed herein, such as I, J and/or L (e.g., corresponding to SEQ ID NOs:2 or 8, SEQ ID NOs:4 or 10, and SEQ ID NOs:6 or 12, respectively) can be incorporated, either alone or in combination with other tropoelastin isoforms into a synthetic elastin polymer with favorable structural and functional properties.

Molecular properties, such as coacervation characteristics and cross-linking, as well as biologic properties, such as elasticity, strength, cell attachment, can be readily determined and characterized using the methods disclosed herein and known to those of ordinary skill. Thus, the properties of any synthetic elastin polymer, such as those including one or more of the novel tropoelastin isoforms disclosed herein, can be determined. Typically, the elastin polymer incorporating the novel tropoelastin isoform is compared to a control or standard. One suitable standard is the tropoelastin A isoform (which is described briefly in Table 2). This tropoelastin isoform can be used as a control against which all other isoform properties are compared, although other standards could be used.

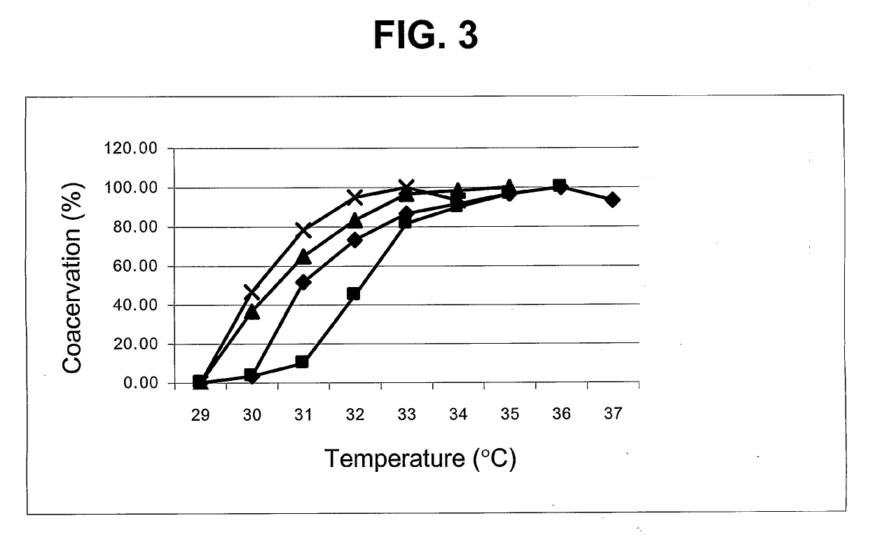

For example, coacervation characteristics, including the temperature and rate at which coacervation occurs, can be determined by a practitioner of ordinary skill in the art. The coacervation characteristics of a tropoelastin isoform, such as any one of isoforms I, J or L, singly or in any combination, can be determined spectrophotometrically (see, e.g., Wu and Weiss, Eur. J. Biochem. 266:308-314, 1999). Coacervate formation causes a solution of the tropoelastin in PBS (or another buffer) to become turbid, and the rate of formation can be followed by measuring the turbidity at 300 nm. For example, to evaluate coacervation properties of a recombinant tropoelastin isoform (such as isoform I, J and/or L, singly or in combination), solutions of tropoelastin isoforms are prepared (e.g., 20 mg/ml) in a buffer approximating physiological conditions (e.g., 10 mM phosphate buffer, pH7.4, 150 mM NaCl) at room temperature, sterile filtered and placed in a spectrophotometer. The change in turbidity of each sample is then recorded until it reaches a maximum. The samples can be cooled on ice to re-dissolve the coacervate, and the solution re-equilibrated at room temperature. This procedure is repeated at temperatures of 28° C. to 40° C. in 1° C. increments to generate a temperature curve. A coacervation curve can then be constructed by calculating the maximum optical density achieved at each temperature as a percentage of the maximum of all readings and graphing the resulting value against the temperature. Changes in coacervation characteristics resulting from differing exon composition of the tropoelastin isoforms can readily be detected using this in vitro coacervation procedure. FIG. 3 illustrates exemplary coaceivation curves for two previously known isoforms (designated A and E in Table 2).

The mechanical properties of synthetic elastin polymers can be analyzed to using a system, such as a Chatillon Vitrodyne V1000 system, that assays tensile strength of the polymer. Methods for determining mechanical properties of a synthetic elastin polymer using this system are described, e.g., in U.S. Pat. No. 6,632,450, which is incorporated herein by reference.

By way of example, the elastin sample is hydrated in buffer prior to testing in the Chatillon Vitrodyn V1000 system with a selected load cell (e.g., 500 g) using tensile grips designed to accept the precut elastin sample. Tension is increased gradually until failure of the elastin polymer (indicated by breakage) at ambient temperature. Force and displacement measurements are acquired at intervals. Engineering stress (force/cross-sectional area) and strain (change in length/original length) are then calculated and plotted. Linear regression of the slope in the stress-strain plots can be used to calculate the elastic modulus (stress/strain). Peak stress and strain are taken as ultimate tensile strength and strain.

An approximately linear curve is typical for an elastic material. Polymers produced from elastin isolated from porcine aorta, which provides a point of comparison for polymers produced from recombinant isoforms, exhibit an ultimate stress in the range of 300-600 kPa; ultimate strain of 100-150%; and an elastic modulus of 300-600 kPa. Polymers with increased cross-linking have a steeper stress/strain curve resulting in an increased elastic modulus as compared to those with poorer cross-linking properties. Thus, this assay can be used to evaluate elasticity and tensile strength of an elastin biomaterial including any tropoelastin isoform (such as isoform I, J or L, or combination thereof with another of isoform I, J and/or L and/or another tropoelastin isoform).

The following examples are provided to illustrate certain particular features and/or embodiments. These examples should not be construed to limit the invention to the particular features or embodiments described.

### EXAMPLES

### Example 1

### Cloning and Characterization of Human Tropoelastin Isoform cDNAs

Previously undescribed human tropoelastin isoforms were identified by screening a human fetal heart cDNA library (Clontech, Palo Alto Calif.). Approximately 1×10⁶ clones of a human fetal heart cDNA library were screened with a 175 bp PCR fragment of human elastin cDNA encompassing exon 20 using standard methods (Sambrook et al., In Molecular Cloning.: A Laboratory Manual, Cold Spring Harbor, N.Y., 1989). The screening yielded 85 positive plaques. Isolated positive clones were further screened by PCR for the presence of the 5′ and 3′ UTRs, to identify full-length clones. Clones that contained full-length transcripts were purified to homogeneity, subcloned into pLITMUS 29 (New England Biolabs), and sequenced with pUC19/M13 forward and reverse primers as well as six internal elastin cDNA-specific sequencing primers to determine isoform composition.

Fifteen tropoelastin full-length clones were sequenced, representing nine different splice variants. The tropoelastin cDNAs representing the nine different splice variants were sub-cloned into pGEX-2T (Amersham Biosciences) for expression in E. coli. The clones were transfected into BL2-CodonPlus (DE3)-RIL cells (Stratagene) as the host to create E. coli cell lines for the expression of recombinant tropoelastin isoforms. Prior to subcloning into the E. coli expression vector, the clones were engineered to remove exon 1, which encodes the secretion signal sequence. The secretion signal peptide is not recognized or cleaved by E. coli, and was therefore removed to prevent it from being incorporated into the recombinant tropoelastin molecule. A methionine residue was added to the 5′ end of exon 2, and the altered inserts were cloned into pGEX-2T (Amersham Biosciences), which produces a GST fusion protein with an amino-terminal GST tag. Typically, expression is increased when tropoelastin is produced as a GST fusion protein rather than tropoelastin alone. The methionine residue separating GST from the amino-terminus of tropoelastin was situated so as to provide a cyanogen bromide cleavage point to facilitate purification. Since there are no other methionine residues in tropoelastin, the final product is unaffected by treatment with cyanogen bromide. Other proteins in the mixture are cleaved, simplifying their removal from the final product.

In addition, human genome databases were accessed through the National Center for Biotechnology Information (NCBI) website, (on the World Wide Web at ncbi.nlm.nih.gov), using the Entrez search function with “homo sapiens elastin” as the search term. All GENBANK® entries and clones from the library screen were analyzed for exon structure based on comparison to a reference cDNA sequence, accession number NM_{—}000501, and to the genomic DNA sequence for human chromosome 7, accession number NT_{—}00758. The Spidey mRNA to genomic alignment program (accessible on the World Wide Web at ncbi.nlm.nih.govlspidey), was used to compare the mRNA transcripts to genomic DNA sequence when analyzing potential alternative splice junctions. The ClustalW Multiple Sequence Alignment tool (accessible on the World Wide Web at clustalw.genome.ip), was used to align the sequences for different mRNA species to facilitate exon definition.

Splice sites were analyzed using the Shapiro-Senaphthy system (Shapiro and Senaphthy, Nucl. Acids Res. 15:7155-7174, 1987). The description and formulas can be found on the World Wide Web at home.snafu.de/probins/Splice?ShapiroSenaphthy.html.

Screening of a human fetal heart cDNA library resulted in the identification of differentially spliced tropoelastin isoform transcripts. Four of these transcripts have been previously reported (Indik et al., PNAS USA 84:5680-5684, 1987; Fazio et al., J Invest Dermatology 91:458-464, 1988), three were inferred from partial clones (Fazio et al., J Invest Dermatology 91:458-464, 1988), and three were previously unrecognized as splice variants and are not found in public databases (i, j and l). Query of the National Center for Biotechnology Information database revealed entries for an additional 10 unique splice variants, as well as showing entries that overlapped with some of the transcripts identified in the library screen and those reported in the literature. Table 2 provides a summary of the exon composition of these 19 isoform transcripts. Each of the isoform transcripts is designated by a letter. The exons that are skipped in (that is, omitted from) each transcript are indicated in the column marked “Skipped Exons”. Although not listed as a skipped exon, none of the transcripts contain exon 22, which has not previously been found to be translated in any tropoelastin isoform. The tissue or cell type from which the transcripts were cloned as cDNA is designated under “Source”.

TABLE 2
Exon composition of 19 verified tropoelastin isoform transcripts.

| Desig- | | | |
|---|---|---|---|
| nation | Skipped Exons | Source | Reference |
| ELN.a | 7A, 26A | SF | Fazio, 1988; |
| | | | Acc: M36860 |
| ELN.b | 7A, 23A | SF | Indik, 1987 |
| ELN.c | 7A, 23A, 26A | SF, FH | Indik, 1987; Maslen |
| ELN.d | 7A, 26A, 32 | SF | Fazio, 1988 |
| ELN.e | 7A, 23, 23A, 26A | SF, FH | Fazio, 1988; Maslen |
| ELN.f | 7A, 23, 23A, 26A, 32 | FH | Indik, 1987; Maslen |
| ELN.g | 7A, 23A, 26A, 32, 33 | FH | Maslen |
| ELN.h | 7A, 23, 23A, 26A, 32, 33 | FH | Maslen |
| ELN.i* | 7A, 13, 23, 23A, 26A | FH | herein |
| ELN.j* | 7A, 19, 23A, 26A, 32 | FH | herein |
| ELN.k | 3, 7A, 23A, 26A, 32 | RT | Acc: BX538199 |
| ELN.l* | 3, 7A, 23, 23A, 26A, 32 | FH | herein |
| ELN.m | 3, 7A, 10, 11, 20A, 23, | CH | AV: ELN.ldec03 |
| | 23A, 26A, 32 | | |
| ELN.n | 5, 7A, 11, 13, 20A, 23, | TC | Acc: AK075554 |
| | 23A, 26A | | |
| ELN.o | 6, 7A, 23A, 26A | CH | AV: ELN.kdec03 |
| ELN.p | 6, 7, 7A, 8, 9, 10, 13, | CH | Acc: AK122731 |
| | 23, 23A, 26A, 32 | | |
| ELN.q | 23A, 26A, 32 | FK, FH | Acc: BX537939; Maslen |
| ELN.r | 3, 23, 23A, 26A | Pl | Acc: BAC86188 |
| ELN.s | 23, 23A, 26A, 32 | SF | Acc: AAC98393 |

*Novel tropoelastin isoforms.
Source designations: FH, fetal heart; SF, skin fibroblast; Pl, placenta; FK, RT, renal tumor; fetal kidney; CH, chondrocyte; TC, teratocarcinoma
References: Acc, GENBANK ® accession number; AV, Ace View designation; Maslen, fetal heart library screen.

The following criteria were used to classify a transcript as being legitimate: 1) confirmation in more than one independent clone; 2) intact 5′ and 3′ untranslated regions; 3) presence of the 5′ secretion signal sequence; 4) absence of premature termination codons; and 5) absence of internal non-exon bound deletions or other sequence rearrangements. In addition, only transcripts that contained exon 36 were considered, as it is known that the domain encoded by exon 36 is required for incorporation of the molecule into the elastin polymer (Kozel et al., J Biol Chem 278:18491-18498, 2003). Transcripts from the library screen and sequences identified in the sequence database were included only if they represented tropoelastin molecules that had the theoretical capacity to participate in elastin biosynthesis. This does not preclude the possibility that there are tropoelastin isoforms with functions other than being incorporated into mature elastin polymer.

### Example 2

### Identification of New ELN Exons

Characterization of tropoelastin transcripts from the fetal heart cDNA library, and those found in public DNA databases, revealed the apparent “insertion” of unexpected sequence, or the partial “deletion” of a previously identified exon sequence in some transcripts. Further investigation revealed differential exon definition in these cases, with alternately spliced contiguous exons revealed as the source of sequence insertion or deletion. All ELN introns are flanked by the canonical 5′gt and 3′ ag dinucleotides that define most mammalian introns. Most of the donor and acceptor splice junctions are considered to be strong based on the Shapiro-Senaphthy score for the consensus sequences (Shapiro and Senaphthy, Nucl. Acids Res. 15:7155-7174, 1987), with 100 being a perfect match of the consensus sequence. This includes the newly defined exons, which are all contiguous with previously defined exons but make use of alternative splice junctions embedded in the coding sequence. The Shapiro-Senaphthy scores for all of the identified splice junctions are listed in Table 3.

TABLE 3
Shapiro-Senapathy scores for the donor and acceptor splice sites
of the introns associated with the verified coding exons for ELN.

| Intron | 5′ donor | 3′ acceptor |
|---|---|---|
| 1 | 95.4 | 98.1 |
| 2 | 81.2 | 92.5 |
| 3 | 90.9 | 92.7 |
| 4 | 94.3 | 90.0 |
| 5 | 96.7 | 91.1 |
| 6 | 88.9 | 92.6 |
| 7 | 82.8 | 90.8 |
| 7A | 82.8 | 78.4 |
| 8 | 82.1 | 95.8 |
| 9 | 91.0 | 93.3 |
| 10 | 82.8 | 99.4 |
| 11 | 92.1 | 96.2 |
| 12 | 92.2 | 95.0 |
| 13 | 88.9 | 96.7 |
| 14 | 95.4 | 93.8 |
| 15 | 88.5 | 87.6 |
| 16 | 80.1 | 86.4 |
| 17 | 88.9 | 97.8 |
| 18 | 90.9 | 96.6 |
| 19 | 88.5 | 94.5 |
| 20 | 69.2 | 84.0 |
| 20A | 68.6 | 84.0 |
| 21 | 91.8 | 77.5 |
| 22 | 90.9 | 91.6 |
| 23 | 92.2 | 94.4 |
| 23A | 92.2 | 74.4 |
| 24 | 96.7 | 86.6 |
| 25 | 75.0 | 91.2 |
| 26 | 66.2 | 96.9 |
| 26A | 62.9 | 96.9 |
| 27 | 88.9 | 93.9 |
| 28 | 88.9 | 90.5 |
| 29 | 83.0 | 88.4 |
| 30 | 91.0 | 92.2 |
| 31 | 88.5 | 93.0 |
| 32 | 84.7 | 93.6 |
| 33 | 82.8 | 91.6 |

Three individual transcripts identified in GENBANK® entries have an additional 15 bp sequence between exons 7 and 8, adding a 5 amino acid residue sequence, APSVP (SEQ ID NO:15), between domains 7 and 8. This is the result of partial retention of the 3′ end of intron 7 due to activation of a cryptic 3′ acceptor splice site at position −17 of intron 7. Although the cryptic acceptor site has a weaker consensus sequence based on the Shapiro Senaphthy score (78.4) than does the constitutive acceptor site (90.8), its alternative usage is apparent in multiple independent transcripts of otherwise varying composition. Both reading frame and codon composition of the terminal split codon are maintained, resulting in the apparent 5 amino acid residue insertion. Using nomenclature consistent with previous terminology, this converted intron sequence has been designated exon 7A, in recognition of its inclusion in apparently viable transcripts as an alternatively spliced exon.

Partial intron retention also occurs with the 3′ end of intron 23, due to activation of a cryptic 3′ acceptor splice site internal to intron 23, thereby creating exon 23A. In this case, the constitutive acceptor splice junction is strong, with a Shapiro and Senaphthy splice score of 94.4, whereas the cryptic splice junction is considerably weaker, with a Shapiro and Senaphthy splice score of 74.4. The 18 nucleotides from the 3′ end of intron 23 are retained, inserting 6 amino acid residues, ALLNLA (SEQ ID NO:16), between domains 23 and 24. This 6 amino acid insertion was originally reported by Fazio et al to be encoded by exon 12A in the previously used gene nomenclature (Fazio et al., J Invest Dermatology 91:458-464, 1988).

Genomic analysis also reveals that there are alternatively spliced products that result from utilization of a cryptic donor splice site in exon 20. This alternative donor site has a Shapiro and Senaphthy splice score of 68.6, which rivals the strength of the more frequently used constitutive donor site that has a score of 69.2. Use of the cryptic splice site maintains the reading frame but removes 26 amino acids. Consequently, what was previously defined as exon 20 is actually two distinct exons, 20 and 20A. If exon 20A is included, then there is a proline between domains 20A and 21. If exon 20A is skipped, then the junctional amino acid residue between domains 20 and 21 is an alanine. It is not clear as to what controls the use of the exon 20A splice junction, but it is interesting to note that there is a coding SNP in exon 20A, a G/A polymorphism resulting in a glycine to serine substitution. Although the amino acid substitution itself is unlikely to influence exon definition, the SNP results in a putative exon splice enhancer consensus sequence. The alteration does not eliminate the potential for this sequence to act as an exon splice enhancer, but rather slightly diminishes the score for the consensus sequence. So far all of the transcripts identified have a glycine in that position when exon 20A is present, but no correlation has been made between exon 20A skipping and the A allele at that position. Changes in amino acid residues as a result of exon skipping are an uncommon event in tropoelastin, as 25 of the 34 junction amino acids encoded by phase I split exons are glycine residues, where the third codon position can be any of the four bases and therefore cannot be altered by exon skipping.

### Example 3

### Alternative splicing of ELN

Analysis of the 19 verified splice variants enumerated in Table 2 indicated that, of the 37 ELN exons, 17 exons are subject to alternative splicing. The different isoform transcripts identified in the fetal heart library screen were all represented by roughly equal numbers of clones, suggesting that no one isoform transcript was predominant in that library.

Exons 3, 5, 6, 7, 7A, 8, 9, 10, 11, 13, 19, 20A, 23, 23A, 26A, 32 and 33 are all involved in exon skipping. Exon 23A and 26A are the most often skipped (that is, omitted) exons. To date, each has only been seen in one isoform transcript, although both exons have been verified in clones from more than one source. Exon 7A is the next most frequently skipped exon, but unlike 23A and 26A it is present in three different isoform transcripts, being utilized with different combinations of other exons and by multiple tissues. By contrast, exons 5, 7, 8, 9, 10 and 19 participate in alternative splicing, but are seldom skipped and are each skipped in only one isoform transcript. Exons 6, 11, 13, 20A and 33 are also skipped with low frequency, but each is missing in more than one isoform transcript. Exons 3, 23 and 32 are active participants in alternative splicing, and are skipped in a wide variety of isoform transcripts and multiple tissues, but are also present in an equally broad range of transcripts. Exon 3 is included in this category even though it had not been previously reported to be alternatively spliced. However, it was found to be skipped in four different isoform transcripts from a variety of sources, indicating that it is alternatively spliced much more frequently than previous studies suggested.

Another long-standing issue is the legitimacy of the putative exon 22. This exon was originally identified in the human ELN gene based on homology with the bovine orthologue, which has an active exon 22. Although it has retained the designation of exon 22, failure to find transcripts that include it suggests that it is not recognized as an exon in the human gene. Analysis of exon 22 and flanking sequence shows that it maintains an open reading frame, and that the flanking introns have identifiable donor and acceptor splice sites. The 3′ acceptor splice site for intron 21 is relatively weak, with a Shapiro Senaphthy score of 77.5, but that alone is probably not sufficient to force constitutive skipping of exon 22. There is a lack of a significant polypyrimidine tract and recognizable branchpoint sequence in intron 21, which could contribute to the failure of exon 22 being identified by the splicing mechanism. A single human transcript reported in GENBANK® (accession number EAHU) includes exon 22 in the cDNA sequence, suggesting that it can be utilized as an exon. The EAHU transcript skips only exon 7A, with all other exons present, including those that are rarely utilized. This record, which has not been updated since June 1999, does not indicate source or verifying clones. Consequently, it is uncertain if this represents a mature, viable transcript or a pre-mRNA that has only been partially spliced. As this is the only report of inclusion of exon 22 in a human transcript, utilization of exon 22 remains to be confirmed. At best, its frequency of inclusion appears to be extremely limited.

Analysis of the 19 confirmed splice variants does not reveal any particular combination of skipped exons. A “full length” transcript that utilizes all 37 confirmed exons has not been identified, indicating that alternative splicing is the norm for ELN, and suggesting that there are many tropoelastin isoforms. The ELN reading frame is always maintained when exons are skipped, so the various transcripts are viable and stable, indicating the potential to be translated into protein. As a result of alternative splicing, the potential for tropoelastin isoforms that vary greatly in size is significant. The longest isoform lacks only exons 7A and 23A, resulting in a protein of 757 amino acids (see Table 2, ELN.b). It is expressed by skin fibroblasts (Fazio et al., J Invest Dermatology 91:458-464, 1988), but has not been reported for other tissues. The shortest transcript found in this study is expressed by chondrocytes (see Table 2, ELN.p), and skips exons 6, 7, 7A, 8, 9, 10, 13, 23, 23A, 26A and 32, encoding a tropoelastin monomer of 570 amino acids.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isolated or recombinant nucleic acid comprising a polynucleotide sequence that encodes a human tropoelastin isoform, wherein the nucleic acid comprises the sequence of exon 20A and excludes a plurality of exons of a human elastin genomic sequence, the plurality of excluded exons selected from the group consisting of exon 3, exon 7A, exon 13, exon 19, exon 22, exon 23, exon 23A, exon 26A, and exon 32.

2. The isolated or recombinant nucleic acid of claim 1, wherein the nucleic acid excludes:
(a) exons 7A, 13, 22, 23, 23A and 26A;
(b) exons 7A, 19, 22, 23A, 26A, and 32; or
(c) exons 3, 7A, 22, 23, 23A, 26A, and 32.

3. The isolated or recombinant nucleic acid of claim 1, wherein the nucleic acid is selected from the group consisting of:
(a) a polynucleotide sequence consisting of exons 2, 3, 4, 5, 6, 7, 8, 9, 10, 11, 12, 14, 15, 16, 17, 18, 19, 20, 20A, 21, 24, 25, 26, 27, 28, 29, 30, 31, 32, 33, and 36 of the human ELN genomic sequence;
(b) a polynucleotide sequence consisting of exons 2, 3, 4, 5, 6, 7, 8, 9, 10, 11, 12, 13, 14, 15, 16, 17, 18, 20, 20A, 21, 23, 24, 25, 26, 27, 28, 29, 30, 31, 33, and 36 of the human ELN genomic sequence;
(c) a polynucleotide sequence consisting of exons 2, 4, 5, 6, 7, 8, 9, 10, 11, 12, 13, 14, 15, 16, 17, 18, 19, 20, 20A, 21, 24, 25, 26, 27, 28, 29, 30, 31, 33, and 36 of the human ELN genomic sequence; and,
(d) a polynucleotide sequence consisting of exon 1 of the human ELN genomic sequence and a polynucleotide sequence of (a), (b) or (c).

4. The isolated or recombinant nucleic acid of claim 1, wherein the nucleic acid comprises a polynucleotide sequence that encodes a polypeptide with the amino acid sequence of SEQ ID NO:2; SEQ ID NO:4, SEQ ID NO:6, SEQ ID NO;8, SEQ ID NO:10, SEQ ID NO: 12, or a variant thereof comprising no more than 10 amino acid deletions, additions or conservative amino acid substitutions.

5. The isolated or recombinant nucleic acid of claim 4, wherein the variant nucleic acid encodes a polypeptide comprising no more than 1 amino acid deletion, addition or conservative amino acid substitution.

6. The isolated or recombinant nucleic acid of claim 1, wherein the nucleic acid comprises a polynucleotide sequence selected from the group consisting of SEQ ID NO:1, SEQ ID NO:3, SEQ ID NO:5, SEQ ID NO:7, SEQ ID NO:9, SEQ ID NO:11, and a variant thereof, which variant hybridizes under high stringency conditions over substantially the entire length to one or more of SEQ ID NO:1, SEQ ID NO:3, SEQ ID NO:5, SEQ ID NO:7, SEQ ID NO:9 or SEQ ID NO:11.

7. An isolated tropoelastin monomer encoded by the nucleic acid of claim 1.

8. The isolated tropoelastin monomer of claim 7, wherein the tropoelastin monomer excludes the amino acids encoded by:
(a) exons 7A, 13, 22, 23, 23A and 26A;
(b) exons 7A, 19, 22, 23A, 26A, and 32; or
(c) exons 3, 7A, 22, 23, 23A, 26A, and 32.

9. The isolated tropoelastin monomer of claim 7, wherein the tropoelastin monomer consists of the amino acids encoded by:
(a) a polynucleotide sequence consisting of exons 2, 3, 4, 5, 6, 7, 8, 9, 10, 11, 12, 14, 15, 16, 17, 18, 19, 20, 20A, 21, 24, 25, 26, 27, 28, 29, 30, 31, 32, 33, and 36 of the human ELN genomic sequence;
(b) a polynucleotide sequence consisting of exons 2, 3, 4, 5, 6, 7, 8, 9, 10, 11, 12, 13, 14, 15, 16, 17, 18, 20, 20A, 21, 23, 24, 25, 26, 27, 28, 29, 30, 31, 33, and 36 of the human ELN genomic sequence;
(c) a polynucleotide sequence consisting of exons 2, 4, 5, 6, 7, 8, 9, 10, 11, 12, 13, 14, 15, 16, 17, 18, 19, 20, 20A, 21, 24, 25, 26, 27, 28, 29, 30, 31, 33, and 36 of the human ELN genomic sequence.

10. The isolated tropoelastin monomer of claim 7, wherein the tropoelastin monomer comprises the amino acid sequence of SEQ ID NO:2; SEQ ID NO:4, SEQ ID NO:6, SEQ ID NO:8, SEQ ID NO:10 or SEQ ID NO:12, or a variant thereof comprising no more than 5 amino acid deletions, additions or conservative amino acid substitutions.

11. The isolated tropoelastin monomer of claim 10, wherein the tropoelastin monomer comprises no more than 1 amino acid deletion, addition or conservative amino acid substitution.

12. The isolated tropoelastin monomer of claim 7, wherein the tropoelastin monomer is encoded by a nucleic acid comprising the polynucleotide sequence of SEQ ID NO:1, SEQ ID NO:3, SEQ ID NO:5, SEQ ID NO:7, SEQ ID NO:9, SEQ ID NO:11, or a variant thereof, which variant hybridizes under high stringency conditions over substantially the entire length to one or more of SEQ ID NO:1, SEQ ID NO:3, SEQ ID NO:5, SEQ ID NO:7, SEQ ID NO:9 or SEQ ID NO:11.

13. A synthetic elastin polymer comprising at least one tropoelastin monomer of claim 7.

14. A synthetic elastin polymer comprising a plurality of different tropoelastin monomers of claim 7.

15. The synthetic elastin polymer of claim 13, wherein the synthetic elastin polymer has at least one of altered coacervation or altered cross-linking as compared to an elastin polymer lacking the at least one tropoelastin monomer.

16. The synthetic elastin polymer of claim 13, wherein the synthetic elastin polymer has at least one improved biological property as compared to an elastin polymer lacking the at least one tropoelastin monomer.

17. The synthetic elastin polymer of claim 16, wherein the synthetic elastin polymer is compared to a second synthetic elastin polymer.

18. The synthetic elastin polymer of claim 16, wherein the synthetic elastin polymer is compared to an elastin polymer comprising isoform A tropoelastin.

19. The synthetic elastin polymer of claim 16, wherein the elastin polymer has at least one of increased tensile strength, increased elasticity, increased chemotaxicity or increased cell-binding as compared to an elastin polymer lacking the at least one tropoelastin monomer.


SEQUENCE LISTING (16 sequences)

SEQ ID NO: 1 - DNA, 2076 bp, Homo sapiens
  CDS (1)..(2076)
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggtttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 2 - protein, 691 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 3 - DNA, 2067 bp, Homo sapiens
  CDS (1)..(2067)
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggagtggcagcaagacctggcttcggattgtctcccattttcccaggtggggcctgcctggggaaagcttgtggccggaagagaaaatga

SEQ ID NO: 4 - protein, 688 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 5 - DNA, 2034 bp, Homo sapiens
  CDS (1)..(2034)
atggcgggtctgacggcggcggccccgcggcccggagtcctcctgctcctgctgtccatcctccacccctctcggcctggaggggtccctggggccattcctggtggagttcctggaggagtcttttatccagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggtttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggagtggcagcaagacctggcttcggattgtctcccattttcccaggtggggcctgcctggggaaagcttgtggccggaagagaaaatga

SEQ ID NO: 6 - protein, 677 aa, Homo sapiens
MAGLTAAAPRPGVLLLLLSILHPSRPGGVPGAIPGGVPGGVFYPALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 7 - DNA, 2004 bp, Homo sapiens
  CDS (1)..(2004)
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggtttagttcctggtgtcggcgtggctcctggagttggcgtggctcctggtgtcggtgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 8 - protein, 667 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGLVPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 9 - DNA, 1995 bp, Homo sapiens
  CDS (1)..(1995)
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggagtggcagcaagacctggcttcggattgtctcccattttcccaggtggggcctgcctggggaaagcttgtggccggaagagaaaatga

SEQ ID NO: 10 - protein, 664 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 11 - DNA, 1962 bp, Homo sapiens
  CDS (1)..(1962)
atggcgggtctgacggcggcggccccgcggcccggagtcctcctgctcctgctgtccatcctccacccctctcggcctggaggggtccctggggccattcctggtggagttcctggaggagtcttttatccagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggtttagttcctggtgtcggcgtggctcctggagttggcgtggctcctggtgtcggtgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggagtggcagcaagacctggcttcggattgtctcccattttcccaggtggggcctgcctggggaaagcttgtggccggaagagaaaatga

SEQ ID NO: 12 - protein, 653 aa, Homo sapiens
MAGLTAAAPRPGVLLLLLSILHPSRPGGVPGAIPGGVPGGVFYPALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGLVPGVGVAPGVGVAPGVGVAPGIGPGGVAAAAKSAAKVAAKAQLRAAAGLGAGIPGLGVGVGVPGLGVGAGVPGLGVGAGVPGFGAVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGVAARPGFGLSPIFPGGACLGKACGRKRK

SEQ ID NO: 13 - DNA, 2274 bp, Homo sapiens
atggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctagtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcaggtgcagatgagggagttaggcggagcctgtcccctgagctcagggaaggagatccctcctcctctcagcacctccccagcaccccctcatcacccagggtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatga

SEQ ID NO: 14 - protein, 748 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGALGVSAAPSVPGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPAGAGIPGAAVPGVVSPEAAAKAAAKAAKYGARPGVGVGGIPTYGVGAGGFPGFGVGVGGIPAGVAGVPGVGGVPGVGGVPGVGISPEAQAAAAAKAAKYGVGTPAAAAAKAAAKAAQFALLNLAGLVPGVGVAPGVGVAPGVGVAPGIGPGGVAAAAKSAAKVAAKAQLRAAAGLGAGIPGLAGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 15 - protein, 5 aa, Homo sapiens
APSVP

SEQ ID NO: 16 - protein, 6 aa, Homo sapiens
ALLNLA